(19) United States
(12) Patent Application Publication
(10) Pub. No.: US 2011/0250138 A1
(43) Pub. Date: Oct. 13, 2011

(54) SINGLE-STRANDED AND DOUBLE-STRANDED OLIGONUCLEOTIDES COMPRISING A METAL-CHELATING LIGAND

(75) Inventors: Yupeng Fan, Arlington, MA (US); Martin Maier, Belmont, MA (US); Rajendra K. Pandey, Framingham, MA (US); Muthiah Manoharan, Weston, MA (US); Kallanthottathil G. Rajeev, Wayland, MA (US)

(73) Assignee: ALNYLAM PHARMACEUTICALS, INC., Cambridge, MA (US)

(21) Appl. No.: 12/671,677

(22) PCT Filed: Jul. 30, 2008
(86) PCT No.: PCT/US2008/071576

§ 371 Date: Jun. 27, 2011

Related U.S. Application Data
(60) Provisional application No. 60/953,359, filed on Aug. 1, 2007.

(51) Int. Cl. A61K 49/00; C07F 5/00

(52) U.S. Cl. 424/9.1; 534/15; 534/16

(57) ABSTRACT

The invention provides modified oligonucleotides of formula (I), comprising at least one metal chelator which provides a powerful tool for study of the pharmacokinetics of siRNA and its correlation with in vivo activity. The chelated metals provide luminescent properties enable detection of the oligonucleotides through the use of time-resolved fluorescent quenching based on energy transfer from the metal ion to a nonfluorescent quencher which can be used as non-isotopic labels of oligonucleotides for diagnostics and evaluation of cellular uptake.

### RELATED APPLICATIONS

This application claims the benefit of priority to U.S. Provisional Patent Application Ser. No. 60/953,359, filed Aug. 1, 2007; the contents of which is hereby incorporated by reference in its entirety.

### BACKGROUND OF THE INVENTION

### Oligonucleotides

Oligonucleotide compounds have important therapeutic applications in medicine. For example, oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleotides that prevent the formation of proteins by gene-silencing.

### Luminescent Lanthanide Chelates

Luminescent lanthanide chelates have highly unusual spectral characteristics that make them useful nonisotopic alternatives to radioactive probes, as alternatives to organic fluorophores, particularly where there are problems of background autofluorescence, and as donors in luminescence resonance energy transfer. These characteristics include millisecond lifetime, sharply spiked emission spectrum (<10 nm fwhm), large Stokes shifts (>150 nm), no self-quenching high quantum yield for lanthanide luminescence (about 1), and solubility.

A commonly used luminescent lanthanide chelate is diethylenetriaminepentaacetic acid (DTPA) covalently attached to an organic chromophore, the latter acting as an antenna or sensitizer to absorb the excitation light and overcome the weak absorbance of the lanthanides. Carbostyril 124 bound to DTPA or to other polyaminocarboxylate chelates can sensitize both europium and terbium, and the complex is an excellent donor in resonance energy transfer experiments.

Conjugation of DTPA-chromophore moieties is most often done through the dianhydride form of DTPA, where one anhydride reacts with an amine-containing chromophore and the other with amine-containing biomolecules. For conjugation, however, the dianhydride has the following disadvantages:

(1) The anhydride is nonspecific in which nucleophilic acyl substitution reactions occur readily. When reacting with DNA, this concern led us to use base-protected DNA in our previous work. However, base deprotection required strongly alkaline conditions, which we found cause significant cleavage of the DTPA-cs124 amide bond, particularly on longer DNAs, and more mild conditions led to questions of incomplete deprotection;

(2) The anhydride is water-labile;

(3) The dianhydride can lead to a number of products, including DTPA disubstituted with chromophore, DTPA attached to the macromolecule with no sensitizer, and macromolecules cross-linked by DTPA; and

(4) The length of the linker arm between DTPA and the macromolecule is fixed.

### Magnetic Resonance Imaging

Magnetic resonance imaging (MRI) has evolved into one of the most powerful techniques in diagnostic clinical medicine and biomedical research by enabling the acquisition of high resolution, three-dimensional images of the distribution of water in vivo. The strong expansion of medical MRI has prompted the development of a new class of pharmacological products, called contrast agents. These agents catalytically shorten the relaxation time of nearby water molecules, thereby enhancing the contrast with background tissues. This utility has prompted research on improved metal-based contrast agents.

Gd(III) is highly paramagnetic with seven unpaired electrons and a long electronic relaxation time, making it an excellent candidate as a relaxation agent. However, the high toxicity in vivo of [Gd(H₂O)₈]³⁺ requires that the metal be complexed by strong organic chelators before it is administered to patients. Current MRI agents require injection of gram quantities of Gd in order to obtain satisfactory contrast in the resulting image. With such large doses required for reasonable image enhancement, most current contrast agents are limited to targeting sites where they can be expected to accumulate in high concentrations, such as in the blood stream.

Therefore, a need exists for improved contrast agents and luminescent lanthanide chelates, which may be rendered site-specific by conjugation to a targeting molecule, such as an oligonucleic acid.

### SUMMARY OF THE INVENTION

One aspect of the present invention relates to a double-stranded oligonucleotide comprising at least one RRMS comprising a metal-chelating ligand, and methods of use thereof In certain embodiments, the metal-chelating ligand is bound to only one of the two oligonucleotide strands comprising the double-stranded oligonucleotide. In certain embodiments, an metal-chelating ligand is bound to both of the oligonucleotide strands comprising the double-stranded oligonucleotide. In certain embodiments, the oligonucleotide strands comprise at least one modified sugar moiety. In certain embodiments, the phosphate linkage in the oligonucleotide has been replaced with a phosphorothioate linkage. In a embodiment, the metal-chelating ligand is a MRI contrast agent or a luminescent lanthanide chelate.

Another aspect of the present invention relates to a single-stranded oligonucleotide comprising at least one metal-chelating ligand, and methods of use thereof In certain embodiments, the oligonucleotide comprises at least one modified sugar moiety. In certain embodiments, the phosphate linkage in the oligonucleotide has been replaced with a phosphorothioate linkage. In a embodiment, the metal-chelating ligand is an MRI contrast agent or a luminescent lanthanide chelate.

### BRIEF DESCRIPTION OF THE FIGURES

FIG. 1 depicts selected embodiments of the invention wherein [A] the oligonucleotide is double stranded, or [B] single stranded. In another embodiment, [C], the oligonucleotide is modified so as to contain an internal Sugar Replacement Monomer Subunit (SRMS).

FIG. 2 depicts one approach to metal chelation to siRNA.

FIG. 3 depicts selected amino-linked oligonucleotides for conjugation.

FIG. 4 depicts selected precursors to amino-linked oligonucleotides for conjugation (X=phosphoroamidite or Icaa-CPG, also known as Long Chain Alkyl Amino Controlled Pore Glass from Fisher Scientific).

FIG. 5 depicts [A] two approaches to the synthesis of metal-chelated oligonucleotides; and [B] two examples of chelators comprising a metal ion and an electrophile.

FIG. 6 depicts [A] [Gd(DOTA)(H₂O)]⁻¹ (Dotarem: Guerbet, Commercial) and related chelators for conjugation; and [B] Gd-TREN-bisHOPO-TAM-EA and related chelator for conjugation.

FIG. 7 depicts one approach to the synthesis of the Gd-TREN-bisHOPO-TAN-EA-related chelator.

FIG. 8 depicts open approach to the conjugation of chelators to oligonucleotides.

FIG. 9 depicts [A] the Alexa Fluor 488 carboxylic acid, succinimidy ester, mixed isomers; and [B] a hexylamino prolinol linker.

FIG. 10 depicts examples of chelators.

FIG. 11 depicts additional examples of chelators, wherein R is a tether to an electrophile (such as an N-hydroxysuccinyl ester or an isothiocyanate).

FIG. 12 depicts 5′- and 3′-amino modified oligonucleotides B and D which were synthesized from corresponding hydroxyprolinol-phthalimido phosphoramidite A and solid support C, respectively (Y is O or S); and a table showing amino linked and Eu(III) labeled oligonucleotides. Note: ‘s’ indicates a phosphorothioate moiety.

FIG. 13 depicts amino-modified and Eu(III)-labeled oligonucletides.

FIG. 14 depicts [A] Gd(III) Chelatedp-SCN-Bn-DOTA and [B] HPLC monitoring of Gd(III) and p-SCN-Bn-DOTA chelation; Buffer A: 50 mM TEAA, pH=7.0; Buffer B: 50 mM TEAA, 70% ACN, pH=7.0; and Gradient: 5% to 80% in 30 minutes.

FIG. 15 depicts HPLC analysis of conjugation of Gd(III) chelated p-SCN-Bn-DOTA to amino labeled Oligonucleotide: Buffer A: 50 mM TEAA, pH=7.0; Buffer B: 50 mM TEAA, 70% ACN, pH=7.0; and Gradient: 5% to 80% in 30 minutes.

FIG. 16 depicts Gd(III) complexed oligonucleotide through p-SCN-Bn-DOTA (N=nucleotide) with a molecular weight of 7696.29 g/mol.

FIG. 17 depicts various oligonucleotides conjugated to a ligand. Note that NA is an oligonucleotide (or a nucleic acid) comprising of either RNA or DNA or chimeric RNA-DNA, DNA-RNA, RNA-DNA-RNA or DNA-RNA-DNA.

### DETAILED DESCRIPTION OF THE INVENTION

Herein it is shown that the ribose sugar of one or more ribonucleotide subunits of an oligonucleotide can be replaced with another moiety, e.g., a non-carbohydrate (preferably cyclic) linker, bearing a metal-chelating ligand. A ribonucleotide subunit in which the ribose sugar of the subunit has been replaced is referred to herein as a ribose replacement modification subunit (RRMS). A cyclic linker may be a carbocyclic ring system, i.e., all ring atoms are carbon atoms, or a heterocyclic ring system, i.e., one or more ring atoms may be a heteroatom, e.g., nitrogen, oxygen, sulfur. The cyclic linker may be a monocyclic ring system, or may contain two or more rings, e.g. fused rings. The cyclic linker may be a fully saturated ring system, or it may contain one or more double bonds.

The linkers further include (i) at least two “backbone attachment points” and (ii) at least one “tethering attachment point.” A “backbone attachment point” as used herein refers to a functional group, e.g. a hydroxyl group, or generally, a bond available for, and that is suitable for incorporation of the linker into the backbone, e.g., the phosphate, or modified phosphate, e.g., sulfur containing, backbone, of a ribonucleic acid. A “tethering attachment point” in some embodiments refers to a constituent ring atom of the cyclic linker, e.g., a carbon atom or a heteroatom (distinct from an atom which provides a backbone attachment point), that connects a metal-chelating ligand. The moiety can be, e.g., an MRI contrast agent or a compound having luminescent properties. In certain embodiments, the selected moiety is connected to the cyclic linker by an intervening tether. Thus, it will often include a functional group, e.g., an amino group, or generally, provide a bond, that is suitable for incorporation or tethering. In certain embodiments, an additional ribonucleotide subunit is modified to contain an additional moiety, such as one which promotes entry into a cell, e.g., a lipophilic moiety, a cholesterol, a folic acid, a carbohydrate, vitamin A, vitamin E, or vitamin K.

Incorporation of one or more RRMSs described herein into an oligonucleotide, such as siRNAs, microRNAs, antagomirs, antisense oligonucleotides, aptamers, decoy nucleic acids, and the like, which is tethered to an MRI contrast agent or a compound having luminescent properties, provides a powerful tool to understand the pharmacokinetics of those nucleic acid analogues and to correlate with in vivo activity. In particular, metal-chelated oligonucleotides can be used to study both uptake and distribution in vivo. Chelation of metals with luminescent properties enables the use of time-resolved fluorescent quenching, based on energy transfer from the metal ion to a nonfluorescent quencher function, as non-isotopic labeling of oligonucleotides for diagnostics and evaluation of cellular uptake. In addition, labeling of oligonucleotides with magnetic resonance imaging (MRI) contrast agents provides a tool to track the distribution of labeled oligonucleotides in animals or patients when it is injected over time.

In certain embodiments, the backbone of the oligonucleotide is modified to improve the stability of the oligonucleotide compound. For example, in certain instances the P═O linkage is changed to a P═S linkage which is not as susceptible to degradation by nucleases in vivo. In certain instances, the 2′-position of the sugar moiety of a nucleotide is converted to an alkyl or heteroalkyl ether. This modification renders the oligonucleotide less prone to nucleolytic degradation.

As mentioned above, the ligand-conjugated oligonucleotides of the invention can be prepared by attaching the ligand to the oligonucleotide through a monomer, e.g., a chemically modified monomer that is integrated into the oligonucleotide agent. In a embodiment, the coupling is by a tether and a linker as described below, and the complex has the formula represented by:

[ligand] --- [tether] --- [linker] --- [oligonucleotide agent]

While, in some cases, embodiments are described with respect to an compound including a ligand, the invention also includes compounds having the structure:

[tether] --- [linker] --- [oligonucleotide]

Methods of making and incorporating the monomers into the oligonucleotide agents and methods of using those agents are included in the invention.

### Oligonucleotides

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as modified oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases. The oligonucleotides of the present invention preferably comprise from about 5 to about 50 nucleosides. It is more preferred that such oligonucleotides comprise from about 8 to about 30 nucleosides, with 15 to 25 nucleosides being particularly preferred.

An oligonucleotide is a polymer of repeating units generically known as nucleotides or nucleosides. An unmodified (naturally occurring) nucleotide has three components: (1) a nitrogenous base linked by one of its nitrogen atoms to (2) a 5-carbon cyclic sugar and (3) a phosphate, esterified to carbon 5 of the sugar. When incorporated into an oligonucleotide chain, the phosphate of a first nucleotide is also esterified to carbon 3 of the sugar of a second, adjacent nucleotide. The “backbone” of an unmodified oligonucleotide consists of (2) and (3), that is, sugars linked together by phosphodiester linkages between the CS (5′) position of the sugar of a first nucleotide and the C3 (3′) position of a second, adjacent nucleotide. A “nucleoside” is the combination of (1) a nucleobase and (2) a sugar in the absence of a phosphate moiety (Kornberg, DNA Replication, W. H. Freeman & Co., San Francisco, 1980, pages 4-7). The backbone of an oligonucleotide positions a series of bases in a specific order; the written representation of this series of bases, which is conventionally written in 5′ to 3′ order, is known as a nucleotide sequence.

Oligonucleotides may comprise nucleoside or nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleosides or nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that an oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA to cause a decrease or loss of function, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

Specific examples of preferred modified oligonucleotides envisioned for use in the oligonucleotides of the present invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified, and in fact more than one of the following modifications may be incorporated in a single oligonucleotide compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included.

Representative United States Patents that teach the preparation of the above phosphorus atom-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248; each of which is hereby incorporated by reference.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439; each of which is hereby incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Representative United States patents that teach the preparation of PNA compounds include, but are not limited to, U.S. Pat. Nos. 5,539,082; 5,714,331; and 5,719,262, each of which is hereby incorporated by reference. Further teaching of PNA compounds can be found in Nielsen et al., Science, 1991, 254, 1497.

Some embodiments of the present invention employ oligonucleotides with phosphorothioate linkages and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the oligonucleotides of the present invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 199#. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents that teach the preparation of certain of the above-noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,808,027; all of which are hereby incorporated by reference.

The oligonucleotides employed in the oligonucleotides of the present invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy [2′-O—CH₂CH₂OCH₃, also known as 2′-O-(2-methoxyethyl) or 2′-MOE] (Martin et al., Helv. Chim. Acta, 1995, 78, 486), i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, the contents of which are incorporated by reference.

Other preferred modifications include T-methoxy 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and those which are disclosed by Ouchi et al. (Drug Design and Discovery 1992, 9:93); Ravasio et al. (J. Org. Chem. 1991, 56:4329); and Delgardo et. al. (Critical Reviews in Therapeutic Drug Carrier Systems 1992, 9:249), each of which is hereby incorporated by reference in its entirety. Further sugar modifications are disclosed by Cook (Anti-Cancer Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the present invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230.

Representative 2′-O-sugar substituent groups are disclosed in U.S. Pat. No. 6,172,209, entitled “Capped 2′-Oxyethoxy Oligonucleotides,” hereby incorporated by reference in its entirety. Representative cyclic 2′-O-sugar substituent groups are also disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the present invention. Representative substitutions for ring O include, but are not limited to, S, CH₂, CHF, and CF₂. See, e.g., Secrist et al., Abstract 21, Program & Abstracts, Tenth International Roundtable, Nucleosides, Nucleotides and their Biological Applications, Park City, Utah, Sep. 16-20, 1992.

Oligonucleotides may also have sugar mimetics, such as cyclobutyl moieties, in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; 5,700,920; and 5,859,221, all of which are hereby incorporated by reference.

Additional modifications may also be made at other positions on the oligonucleotide, particularly the 3′ position of the sugar on the 3′ terminal nucleotide. For example, one additional modification of the oligonucleotides of the present invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, each of which is hereby incorporated by reference.

The present invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

The present invention further encompasses oligonucleotides employing ribozymes. Synthetic RNA molecules and derivatives thereof that catalyze highly specific endoribonuclease activities are known as ribozymes. (See, generally, U.S. Pat. No. 5,543,508 to Haseloff et al., and U.S. Pat. No. 5,545,729 to Goodchild et al.) The cleavage reactions are catalyzed by the RNA molecules themselves. In naturally occurring RNA molecules, the sites of self-catalyzed cleavage are located within highly conserved regions of RNA secondary structure (Buzayan et al., Proc. Natl. Acad. Sci. U.S.A., 1986, 83, 8859; Forster et al., Cell, 1987, 50, 9). Naturally occurring autocatalytic RNA molecules have been modified to generate ribozymes which can be targeted to a particular cellular or pathogenic RNA molecule with a high degree of specificity. Thus, ribozymes serve the same general purpose as antisense oligonucleotides (i.e., modulation of expression of a specific gene) and, like oligonucleotides, are nucleic acids possessing significant portions of single-strandedness. That is, ribozymes have substantial chemical and functional identity with oligonucleotides and are thus considered to be equivalents for purposes of the present invention.

In certain instances, the oligonucleotide may be further modified to comprise a non-metal-chelating ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate.

Alternatively, the molecule being conjugated may be converted into a building block, such as a phosphoramidite, via an alcohol group present in the molecule or by attachment of a linker bearing an alcohol group that may be phosphitylated.

Importantly, each of these approaches may be used for the synthesis of oligonucleotides comprising a non-natural nucleobase.

The oligonucleotide agents of the invention include nucleic acid targeting (NAT) oligonucleotide agents and protein-targeting (PT) oligonucleotide agents. NAT and PT oligonucleotide agents refer to single-stranded oligomers or polymers of ribonucleic acid (RNA) or deoxyribonucleic acid (DNA) or combined (chimeric) modifications of DNA and RNA. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as enhanced cellular uptake, enhanced affinity for nucleic acid target, and/or increased stability in the presence of nucleases. NATs designed to bind to specific RNA or DNA targets have substantial complementarity, e.g., at least 70, 80, 90, or 100% complementary, with at least 10, 20, or 30 or more bases of a target nucleic acid, and include antisense RNAs, miRNAs, and other non-duplex structures which can modulate expression. Other NAT oligonucleotide agents include external guide sequence (EGS) oligonucleotides (oligozymes), DNAzymes, and ribozymes. These NATs may or may not bind via Watson-Crick complementarity to their targets. PT oligonucleotide agents bind to protein targets, preferably by virtue of three-dimensional interactions, and modulate protein activity. They include decoy RNAs, aptamers, and the like.

The single-stranded oligonucleotide compounds of the invention preferably comprise from about 8 to about 50 nucleobases (i.e. from about 8 to about 50 linked nucleosides). NAT oligonucleotide agents are preferably about 15 nucleotides long, or more preferably about 30 nucleotides long. PT oligonucleotide agents are preferably about 18 nucleotides long, or more preferably about 23 nucleotides long. Particularly preferred compounds are miRNAs and antisense oligonucleotides, even more preferably those comprising from about 12 to about 30 nucleobases.

### MicroRNAs

The oligonucleotide agents include microRNAs (miRNAs). MicroRNAs are small noncoding RNA molecules that are capable of causing post-transcriptional silencing of specific genes in cells such as by the inhibition of translation or through degradation of the targeted mRNA. A miRNA can be completely complementary or can have a region of noncomplementarity with a target nucleic acid, consequently resulting in a “bulge” at the region of non-complementarity. The region of non-complementarity (the bulge) can be flanked by regions of sufficient complementarity, preferably complete complementarity to allow duplex formation. Preferably, the regions of complementarity are at least 8 to 10 nucleotides long (e.g., 8, 9, or 10 nucleotides long). A miRNA can inhibit gene expression by repressing translation, such as when the microRNA is not completely complementary to the target nucleic acid, or by causing target RNA degradation, which is believed to occur only when the miRNA binds its target with perfect complementarity. The invention also includes double-stranded precursors of miRNAs that may or may not form a bulge when bound to their targets.

A miRNA or pre-miRNA can be about 18-100 nucleotides in length, and more preferably from about 18-80 nucleotides in length. Mature miRNAs can have a length of about 19-30 nucleotides, preferably about 21-25 nucleotides, particularly 21, 22, 23, 24, or 25 nucleotides. MicroRNA precursors can have a length of about 70-100 nucleotides and have a hairpin conformation. MicroRNAs can be generated in vivo from pre-miRNAs by enzymes called Dicer and Drosha that specifically process long pre-miRNA into functional miRNA. The microRNAs or precursor miRNAs featured in the invention can be synthesized in vivo by a cell-based system or can be chemically synthesized. MicroRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. Methods of synthesis and chemical modifications are described in greater detail below.

In particular, an miRNA or a pre-miRNA featured in the invention can have a chemical modification on a nucleotide in an internal (i.e., non-terminal) region having noncomplementarity with the target nucleic acid. For example, a modified nucleotide can be incorporated into the region of a miRNA that forms a bulge. The modification can include a ligand attached to the miRNA, e.g., by a linker. The modification can, for example, improve pharmacokinetics or stability of a therapeutic miRNA, or improve hybridization properties (e.g., hybridization thermodynamics) of the miRNA to a target nucleic acid. In some embodiments, it is preferred that the orientation of a modification or ligand incorporated into or tethered to the bulge region of a miRNA is oriented to occupy the space in the bulge region. This orientation facilitates the improved hybridization properties or an otherwise desired characteristic of the miRNA. For example, the modification can include a modified base or sugar on the nucleic acid strand or a ligand that functions as an intercalator. These are preferably located in the bulge. The intercalator can be an aromatic, e.g., a polycyclic aromatic or heterocyclic aromatic compound. A polycyclic intercalator can have stacking capabilities, and can include systems with 2, 3, or 4 fused rings. Universal bases can also be incorporated into the miRNAs.

In one embodiment, an miRNA or a pre-miRNA can include an aminoglycoside ligand, which can cause the miRNA to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. Neo-S-acridine, an acridine analog, has an increased affinity for the HIV Rev-response element (RRE). In some embodiments, the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

A miRNA or a pre-miRNA can be designed and synthesized to include a region of noncomplementarity (e.g., a region that is 3, 4, 5, or 6 nucleotides long) flanked by regions of sufficient complementarity to form a duplex (e.g., regions that are 7, 8, 9, 10, or 11 nucleotides long). For increased nuclease resistance and/or binding affinity to the target, the miRNA sequences can include 2′-O-methyl, 2′-fluorine, 2′-O-methoxyethyl, 2′-O-aminopropyl, 2′-amino, and/or phosphorothioate linkages. The inclusion of furanose sugars in the oligonucleotide backbone can also decrease endonucleolytic cleavage. An miRNA or a pre-miRNA can be further modified by including a 3′-cationic group, or by inverting the nucleoside at the 3′-terminus with a 3′-3′ linkage. In another alternative, the 3′-terminus can be blocked with an aminoalkyl group, e.g., a 3′-C5-aminoalkyl dT. Other 3′-conjugates can inhibit 3′-5′ exonucleolytic cleavage. While not being bound by theory, a 3′-conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

In one embodiment, a miRNA or a pre-miRNA includes a modification that improves targeting, e.g. a targeting modification described above. Examples of modifications that target miRNA molecules to particular cell types include carbohydrate sugars such as galactose, N-acetylgalactosamine, mannose; vitamins such as folates; other ligands such as RGDs and RGD mimics; and small molecules including naproxen, ibuprofen or other known protein-binding molecules.

A miRNA or a pre-miRNA can be constructed using chemical synthesis and/or enzymatic ligation reactions using procedures known in the art. For example, a miRNA or a pre-miRNA can be chemically synthesized using naturally occurring nucleotides or variously modified nucleotides designed to increase the biological stability of the molecules or to increase the physical stability of the duplex formed between the miRNA or a pre-miRNA and target nucleic acids, e.g., phosphorothioate derivatives and acridine substituted nucleotides can be used. Other appropriate nucleic acid modifications are described herein. Alternatively, the miRNA or pre-miRNA nucleic acid can be produced biologically using an expression vector into which a nucleic acid has been subcloned in an antisense orientation, i.e., RNA transcribed from the inserted nucleic acid will be of an antisense orientation to a target nucleic acid of interest.

### Antisense Nucleic Acid Sequences

The single-stranded oligonucleotide agents featured in the invention include antisense nucleic acids. An “antisense” nucleic acid includes a nucleotide sequence that is complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. Accordingly, an antisense nucleic acid can form hydrogen bonds with a sense nucleic acid target.

Given a coding strand sequence such as the sequence of a sense strand of a cDNA molecule, antisense nucleic acids can be designed according to the rules of Watson and Crick base pairing. The antisense nucleic acid molecule can be complementary to a portion of the coding or noncoding region of an RNA, e.g., a pre-mRNA or mRNA. For example, the antisense oligonucleotide can be complementary to the region surrounding the translation start site of a pre-mRNA or mRNA, e.g., the 5′ UTR. An antisense oligonucleotide can be about 10 to 25 nucleotides in length (e.g., 11, 12, 13, 14, 15, 16, 18, 19, 20, 21, 22, 23, or 24 nucleotides in length). An antisense oligonucleotide can also be complementary to a miRNA or pre-miRNA.

An antisense nucleic acid can be constructed using chemical synthesis and/or enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and target nucleic acids, e.g., phosphorothioate derivatives and acridine substituted nucleotides can be used. Other appropriate nucleic acid modifications are described herein. Alternatively, the antisense nucleic acid can be produced biologically using an expression vector into which a nucleic acid has been subcloned in an antisense orientation, i.e., RNA transcribed from the inserted nucleic acid will be of an antisense orientation to a target nucleic acid of interest.

An antisense agent can include ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA, and prevent access of the translation machinery to the target RNA transcript, thereby preventing protein synthesis. An antisense molecule including only deoxyribonucleotides, or deoxyribonucleotides and ribonucleotides, e.g., DNA sequence flanked by RNA sequence at the 5′ and 3′ ends of the antisense agent, can hybridize to a complementary RNA, and the RNA target can be subsequently cleaved by an enzyme such as RNAse H. Degradation of the target RNA prevents translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAse H activity is desired.

For increased nuclease resistance, an antisense agent can be further modified by inverting the nucleoside at the 3′-terminus with a 3′-3′ linkage. In another alternative, the 3′-terminus can be blocked with an aminoalkyl group. In certain instances, the antisense oligonucleotide agent includes a modification that improves targeting, e.g. a targeting modification.

### Decoy Nucleic Acids

An oligonucleotide agent featured in the invention can be a decoy nucleic acid such as decoy RNA. A decoy nucleic acid resembles a natural nucleic acid, but is modified to inhibit or interrupt the activity of the natural nucleic acid. For example, a decoy RNA can mimic the natural binding domain for a ligand, and compete with natural binding target for the binding of a specific ligand. It has been shown that over-expression of HIV trans-activation response (TAR) RNA can act as a “decoy” and efficiently bind HIV that protein, thereby preventing it from binding to TAR sequences encoded in the HW RNA. In one embodiment, a decoy RNA includes a modification that improves targeting. The chemical modifications described above for miRNAs and antisense RNAs, and described elsewhere herein, are also appropriate for use in decoy nucleic acids.

### Aptamers

Oligonucleotide agents of the invention also include aptamers. An aptamer binds to a non-nucleic acid ligand, such as a small organic molecule or protein, e.g., a transcription or translation factor, and subsequently modifies its activity. An aptamer can fold into a specific structure that directs the recognition of the targeted binding site on the non-nucleic acid ligand. An aptamer can contain any of the modifications described herein. In certain instances, the aptamer includes a modification that improves targeting, e.g., a targeting modification. The chemical modifications described above for miRNAs and antisense RNAs, and described elsewhere herein, are also appropriate for use in decoy nucleic acids.

### Additional Features of the Oligonucleotides of the Invention

An oligonucleotide agent that is NAT (“nucleic acid targeting”) includes a region of sufficient complementarity to the target gene, and is of sufficient length in terms of nucleotides, such that the oligonucleotide agent forms a duplex with the target nucleic acid. The oligonucleotide agent can modulate the function of the targeted molecule. For example, when the targeted molecule is an mRNA or pre-mRNA, the NAT can inhibit gene expression; when the target is an miRNA, the NAT will inhibit the miRNA function and will thus up-regulate expression of the mRNAs targeted by the particular miRNA. Alternatively, when the target is a region of a pre-mRNA that affects splicing, the NAT can alter the choice of splice site and thus the mRNA sequence; when the NAT functions as an miRNA, expression of the targeted mRNA is inhibited. For ease of exposition the term nucleotide or ribonucleotide is sometimes used herein in reference to one or more monomeric subunits of an oligonucleotide agent. It will be understood that the term “ribonucleotide” or “nucleotide” can, in the case of a modified RNA or nucleotide surrogate, also refer to a modified nucleotide, or surrogate replacement moiety at one or more positions.

A NAT oligonucleotide agent is, or includes, a region that is at least partially, and in some embodiments fully, complementary to the target RNA. It is not necessary that there be perfect complementarity between the oligonucleotide agent and the target, but the correspondence must be sufficient to enable the oligonucleotide agent, or a cleavage product thereof, to modulate (e.g., inhibit) target gene expression.

The oligonucleotide agent will preferably have one or more of the following properties: (1) it will have a 5′ modification that includes one or more phosphate groups or one or more analogs of a phosphate group; (2) it will, despite modifications even to a very large number of bases, specifically base pair and form a duplex structure with a homologous target RNA of sufficient thermodynamic stability to allow modulation of the activity of the targeted RNA; and (3) it will, despite modifications even to a very large number, or all of the nucleosides, still have “RNA-like” properties, i.e., it will possess the overall structural, chemical and physical properties of an RNA molecule, even though not exclusively, or even partly, of ribonucleotide-based content. For example, all of the nucleotide sugars can contain a 2′-fluoro group in place of 2′-hydroxyl group. This deoxyribonucleotide-containing agent can still be expected to exhibit RNA-like properties. While not wishing to be bound by theory, the electronegative fluorine prefers an axial orientation when attached to the C2′-position of ribose. This spatial preference of fluorine can force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. Generally, it is preferred that a modified moiety at the 2′-sugar position will be able to enter into hydrogen-bonding which is more characteristic of the 2′-OH moiety of a ribonucleotide than the 2′-H moiety of a deoxyribonucleotide. A preferred oligonucleotide agent will: exhibit a C_{3′}-endo pucker in all, or at least about 50, 75, 80, 85, 90, or 95% of its sugars; exhibit a C_{3′}-endo pucker in a sufficient amount of its sugars that it can give rise to the RNA-characteristic A-family-type helix; will generally have no more than about 20, 10, 5, 4, 3, 2, or 1 sugar which is not a C_{3′}-endo pucker structure. In certain instances, oligonucleotide will exhibit C_{3′}-endo suger pucker and be modified at the 2′-position. Exemplary modifications include 2′-OH, 2′-O-Me, 2′-O-methoxyethyl, 2′-O-aminopropyl, 2′-F, 2′-O—CH₂—CO—NHMe, 2′-O—CH₂—CH₂—O—CH₂—CH₂—N(Me)₂, and LNA. In certain instances, regardless of the nature of the modification, and even though the oligonucleotide agent can contain deoxynucleotides or modified deoxynucleotides, it is preferred that DNA molecules, or any molecule in which more than 50, 60, or 70% of the nucleotides in the molecule are deoxyribonucleotides, or modified deoxyribonucleotides which are deoxy at the 2′ position, are excluded from the definition of oligonucleotide agent. Some preferred 2′-modifications with of sugar moieties exhibiting C2′-endo sugar pucker include 2′-H, 2′-Me, 2′-S-Me, 2′-Ethynyl, and 2′-ara-F. Additional sugar modifications include L-sugars and 2′-5′-linked sugars.

As used herein, “specifically hybridizable” and “complementary” are terms that are used to indicate a sufficient degree of complementarity such that stable and specific binding occurs between a compound of the invention and a target RNA molecule. This nomenclature also applies to instances when NAT oligonucleotides agents bind to target RNAs. Specific binding requires a sufficient lack of complementarity to non-target sequences under conditions in which specific binding is desired, i.e., under physiological conditions in the case of in vivo assays or therapeutic treatment, or in the case of in vitro assays, under conditions in which the assays are performed. It has been shown that a single mismatch between targeted and non-targeted sequences are sufficient to provide discrimination for siRNA targeting of an mRNA (Brummelkamp et al., Cancer Cell, 2002, 2:243).

In certain instances, a NAT oligonucleotide agent is “sufficiently complementary” to a target RNA, such that the oligonucleotide agent inhibits production of protein encoded by the target mRNA. The target RNA can be a pre-mRNA, mRNA, or miRNA endogenous to the subject. In another embodiment, the oligonucleotide agent is “exactly complementary” (excluding the SRMS containing subunit(s)) to a target RNA, e.g., the target RNA and the oligonucleotide agent can anneal to form a hybrid made exclusively of Watson-Crick base pairs in the region of exact complementarity. A “sufficiently complementary” target RNA can include a region (e.g., of at least about 7 nucleotides) that is exactly complementary to a target RNA. Moreover, in some embodiments, the oligonucleotide agent specifically discriminates a single-nucleotide difference. In this case, the oligonucleotide agent only down-regulates gene expression if exact complementary is found in the region the single-nucleotide difference.

Oligonucleotide agents discussed include otherwise unmodified RNA and DNA as well as RNA and DNA that have been modified. Examples of modified RNA and DNA include modifications to improve efficacy and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The literature has referred to rare or unusual, but naturally occurring, RNAs as modified RNAs. See Limbach et al. Nucleic Acids Res. 1994, 22, 2183-2196. Such rare or unusual RNAs, often termed modified RNAs, are typically the result of a post-transcriptional modification and are within the scope of the term unmodified RNA as used herein. Modified RNA as used herein refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone.

### Sugar Replacement Monomer Subunits (SRMS)

As discussed above, a nucleotide subunit in which the sugar of the subunit has been so replaced is referred to herein as a sugar replacement modification subunit (SRMS). The SRMS includes two “backbone attachment points” (hydroxyl groups), a “tethering attachment point,” and a ligand, which is connected indirectly to the SRMS via an intervening tether. The SRMS may be the 5′-or 3′-terminal subunit of the oligonucleotide agent and located adjacent to two or more unmodified or modified ribonucleotides. Alternatively, the SRMS may occupy an internal position located adjacent to one or more unmodified or modified ribonucleotides. More than one SRMS may be present in an oligonucleotide agent. Preferred positions for inclusion of a SRMS tethered to a moiety (e.g., a metal-chelating ligand) are at the 3′-terminus, the 5′-terminus, or at an internal position.

In certain embodiments, the SRMS (“linker”) is a pyrroline-based linker, a 3-hydroxypyrline-based linker, a piperidine based linker, morpholine-based liner, a piperazine-based linker, a decalin-based linker, or a indene-based linker. See, for example, Manoharan, M. et al. U.S. Patent Application Publication No. 2005/107325, hereby incorporated by reference. In certain embodiments, the linker is a pyrroline-based linker In certain embodiments of the present disclosure linker is a dihydroxypyrroline-based linker.

### Preparation of Oligonucleotides

The oligonucleotide compounds of the invention can be prepared using solution-phase or solid-phase organic synthesis. Organic synthesis offers the advantage that the oligonucleotide strands comprising an metal-chelating ligand tethered to a nucleobase can be easily prepared. The double-stranded oligonucleotide compounds of the invention comprising an metal-chelating ligand tethered to a nucleobase may be prepared using a two-step procedure. First, the individual strands of the double-stranded molecule are prepared separately. Then, the component strands are annealed.

The ligand-conjugated oligonucleotides of the invention may be synthesized by the use of an oligonucleotide that bears a pendant reactive functionality, such as that derived from the attachment of a linking molecule onto the oligonucleotide. This reactive oligonucleotide may be reacted directly with commercially-available ligands, ligands that are synthesized bearing any of a variety of protecting groups, or ligands that have a linking moiety attached thereto. The methods of the present invention facilitate the synthesis of ligand-conjugated oligonucleotides by the use of, in some embodiments, nucleoside monomers that have been appropriately conjugated with ligands and that may further be attached to a solid-support material. Such ligand-nucleoside conjugates, optionally attached to a solid-support material, are prepared according to some embodiments of the methods of the present invention via reaction of a selected serum-binding ligand with a linking moiety located on the 5′ position of a nucleoside or oligonucleotide. In certain instances, an oligonucleotide bearing an metal-chelating ligand attached to the 3′-terminus of the oligonucleotide is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

Teachings regarding the synthesis of particular modified oligonucleotides may be found in the following U.S. patents: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having 13-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; U.S. Pat. Nos. 6,262,241, and 5,459,255, drawn to, inter alia, methods of synthesizing 2′-fluoro-oligonucleotides.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a embodiment, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

The incorporation of a 2′-O-methyl, 2′-O-ethyl, 2′-O-propyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-O-methyl, 2′-O-ethyl, 2′-O-propyl, 2′-O-aminoalkyl, 2′-O-allyl or 2′-deoxy-2′-fluoro group.

In some embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a embodiment, ligand molecules may be conjugated to oligonucleotides at the 5′-position by the use of a ligand-nucleoside phosphoramidite wherein the ligand is linked to the 5′-hydroxy group directly or indirectly via a linker Such ligand-nucleoside phosphoramidites are typically used at the end of an automated synthesis procedure to provide a ligand-conjugated oligonucleotide bearing the ligand at the 5′-terminus.

In one embodiment of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the present invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, for example, are disclosed by Beaucage et al. (Tetrahedron, 1992, 48:2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y, 1991.

Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821; Verhart and Tesser, Rec. Tray. Chim. Pays-Bas, 1987, 107:621).

Additional amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the present invention.

Many solid supports are commercially available and one of ordinary skill in the art can readily select a solid support to be used in the solid-phase synthesis steps. In certain embodiments, a universal support is used. A universal support allows for preparation of oligonucleotides having unusual or modified nucleotides located at the 3′-terminus of the oligonucleotide. Universal Support 500 and Universal Support II are universal supports that are commercially available from Glen Research, 22825 Davis Drive, Sterling, Va. For further details about universal supports see Scott et al., Innovations and Perspectives in solid-phase Synthesis, 3rd International Symposium, 1994, Ed. Roger Epton, Mayflower Worldwide, 115-124]; Azhayev, A. V. Tetrahedron 1999, 55, 787-800; and Azhayev and Antopolsky Tetrahedron 2001, 57, 4977-4986. In addition, it has been reported that the oligonucleotide can be cleaved from the universal support under milder reaction conditions when oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

### Metal-Chelating Ligands

Preferred moieties are metal-chelating ligands, which are coupled, preferably covalently, either directly or indirectly via an intervening tether, to the SRMS carrier. In embodiments, the metal-chelating ligand is attached to the carrier via an intervening tether. As discussed above, the ligand or tethered ligand may be present on the SRMS monomer when the SRMS monomer is incorporated into the growing strand. In some embodiments, the ligand may be incorporated into a “precursor” SRMS after a “precursor” SRMS monomer has been incorporated into the growing strand. For example, an SRMS monomer having an amino-terminated tether (i.e., having no associated ligand), or TAP—(CH₂)ₙNH₂ may be incorporated into a growing oligonucleotide strand. In a subsequent operation, a metal-chelating ligand having an electrophilic group can subsequently be attached to the precursor SRMS by coupling the electrophilic group of the ligand with a terminal nucleophilic group of the precursor SRMS tether. Representative electrophilic groups include pentafluorophenyl esters or an aldehyde. Other electrophilic groups amenable to the present invention can be readily determined by one of ordinary skill in the art.

In certain embodiments, the metal-chelating ligand is selected from cyclic and acyclic poly(amino-carboxylates) such as DTPA, DOTA, DO3A, 2-benzyl-DOTA, alpha-(2-phenyl)-1,4,7,10-tetraazacyclo-dodecane-1-acetic-4,7,10-tris-(methyl acetic) acid, 2-benzyl-cyclohexyldiethylenetriamine-pentaacetic acid, 2-benzyl-6-methyl-DTPA, and 6,6″-bis-[N,N,N″,N″-tetra(carboxymethyl)-aminomethyl]-4′-(3-amino-4-methyoxyphenyl)-2,2′,6′,2″-terpyridine. Procedures for synthesizing these chelators that are not commercially available can be found in J. Nucl. Med. 1990, 31, 473; Bioconjugate Chem. 1991, 2, 187; Nucleic Acid Research 1991, 19, 1057; J. Chem. Soc. Perkin Trans. 1992, 1, 1175; Bioconjugate Chem. 1997, 8, 127; Bioconjugate Chem. 2005, 16, 700; Bioconjugate Chem. 2006, 17, 571; U.S. Pat. No. 5,064,956, hereby incorporated by reference; U.S. Pat. No. 4,859,777, hereby incorporated by reference; U.S. Pat. App. Pub. No. 2003/0235843-A1, hereby incorporated by reference; and U.S. Pat. App. Pub. No. 2007/0014721-A1, hereby incorporated by reference.

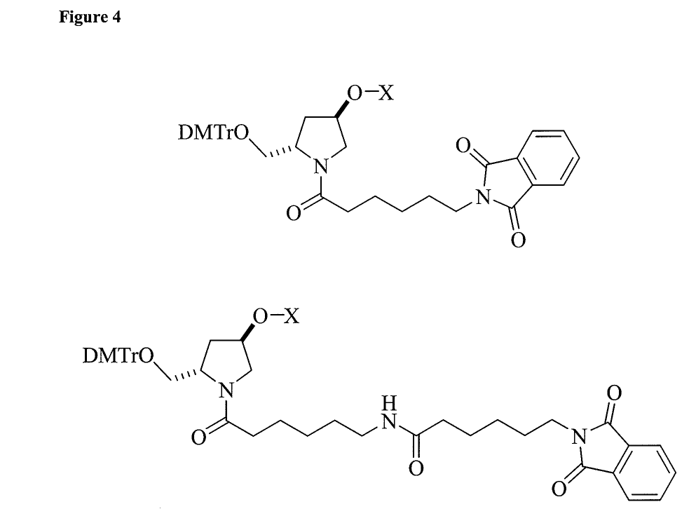

In certain embodiments, the metal-chelating ligand is selected from hydroxypyridinone (HOPO) and related ligands, including mixed or heteropodal complexes and some complexes with varying caps, such as TREN-1-Me-3,2-HOPO, TREN-MOE-3,2-HOPO, TREN-Me₂-5,4-HOPY, TREN-6-Me-3,2-HOPO, TREN-1,2-HOPO, TREN-1,2,3-HOPO, Ser₃-TREN-Me-3,2-HOPO, TRPN-1-Me-3,2-HOPO, TREN-bisHOPO-TAM, TREN-bisHOPO-SAM, TREN-bisHOPO-IAM, TREN-bis(6-Me-HOPO)-TAM-tri, TREN-HOPO-bisTAM-EA, and TREN-TAM-EA. For a recent review of hydroxypyridinone (HOPO) and related ligands, see Bioconjugate Chem. 2005, 16, 3 (in particular, FIG. 4). Procedures for synthesizing these chelators that are not commercially available can be found in Bioconjugate Chem. 2005, 16, 3; J. Med. Chem. 2005, 48, 3874; J. Am. Chem. Soc. 2006, 128, 2222; J. Am. Chem. Soc. 1995, 117, 7245; J. Am. Chem. Soc. 2000, 122, 11228; J. Am. Chem. Soc. 2005, 127, 504; J. Am. Chem. Soc. 2006, 128, 5344; and J. Am. Chem. Soc. 2001, 123, 10758.

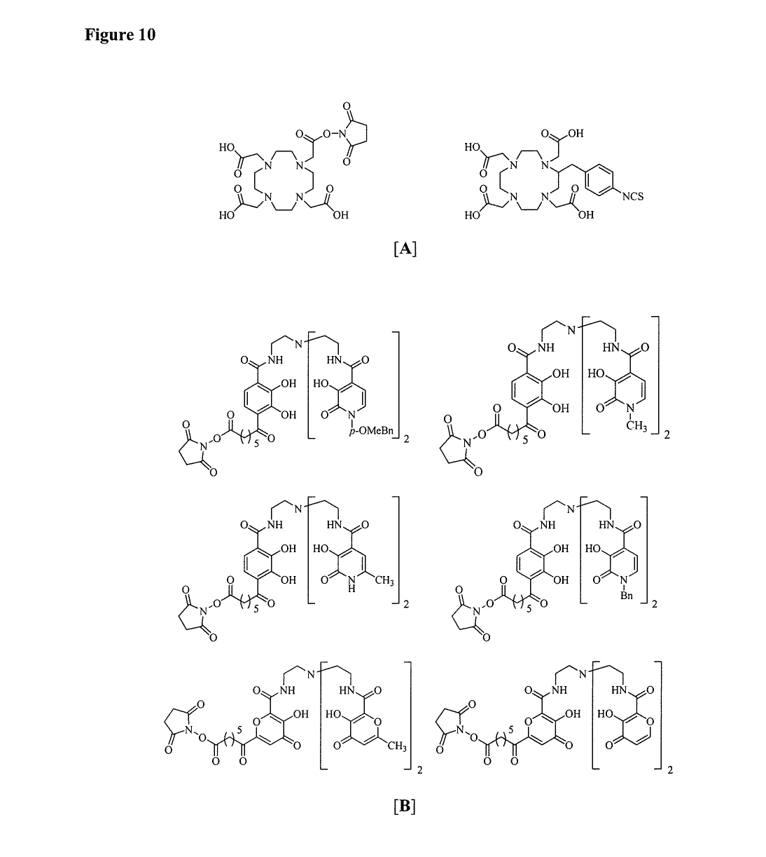

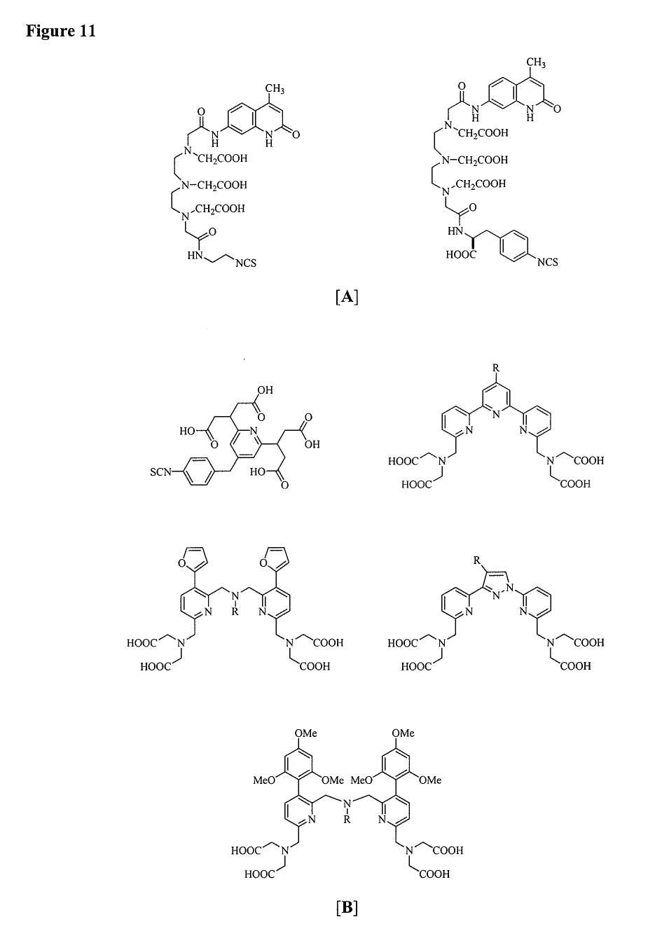

A few examples of metal-chelating ligands, bearing electrophilic groups, are shown in FIGS. 10 and 11.

### Metal Ions

In certain embodiments, the metal-chelating ligand comprises a metal ion. In certain embodiments, the metal is a paramagnetic metal ion. In certain embodiments, the metal ion is Gd(III), Mn(II), Mn(III), Cr(II), Cr(III), Cu(II), Fe (III), Pr(III), Nd(III), Sm(III), Tb(III), Yb(III) Dy(III), Ho(III), Eu(II), Eu(III), or Er(III). In certain embodiments, the metal ion is Gd(III) or Eu(III).

### Tethers

The term “tether,” as used herein, refers to a portion of a molecule that serves as a spacer between two other portions of the molecule. Tethers may also serve other functions as described herein.

In one embodiment of the invention, the metal-chelating ligand is attached to an oligonucleotide via a tether and linking group, to form a ligand-conjugated oligonucleotide. In certain embodiments, the tether selected from the group consisting of alkylene, alkenylene, arylene, heteroalkylene, arylalkylene, heterocyclylene, or a combination thereof. In certain embodiments, the tether is alkylene. In certain embodiments, the tether is alkylene-N(H)C(═O)-alkylene.

In certain embodiments, tethers of the invention include, but are not limited to, 6-aminoalkoxy linkers, 6-aminoalkylamino linkers, cysteamine, heterobifunctional linkers, homobifunctional linkers, and a universal tether (derived from 3-dimethoxytrityloxy-2-aminopropanol). An example of a tether for the synthesis of ligand conjugated oligonucleotides of the invention is a 6-aminohexyloxy group. A variety of heterobifunctional and homobifunctional tethers are available from Pierce Co. (Rockford, Ill.). Such heterobifunctional and homobifunctional tethers are particularly useful in conjunction with the 6-aminoalkoxy and 6-aminoalkylamino moieties to form extended tethers useful for linking ligands to a nucleoside. Further useful tethers that are commercially available are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, while the Y-Amino-Modifier is also available from Clontech Laboratories Inc. (Palo Alto, Calif.). In addition, a nucleotide analog bearing a tether pre-attached to the nucleoside is commercially available from Glen Research Corporation under the tradename “Amino-Modifier-dT.” This nucleoside-tether reagent, a uridine derivative having an [N-(7-trifluoroacetylamino-heptyl)-3-acrylamido] substituent group at the 5-position of the pyrimidine ring, is synthesized as per the procedure of Jablonski et al. (Nucleic Acid Research, 1986, 14:6115).

### Oligonucleotides Bearing at Least One Metal-Chelating Ligand

The compounds of the invention relate to an oligonucleotide bearing at least one metal-chelating ligand. The metal-chelating ligand may render the oligonucleotide trackable in vivo. In certain embodiments, the compounds of the invention relate to a double-stranded oligonucleotide sequence, wherein the metal-chelating ligand is bound to only one of the two strands. In certain embodiments, the compounds of the invention relate to a double-stranded oligonucleotide sequence, wherein at least one metal-chelating ligand is bound to both of the strands. In certain embodiments, the backbone of the oligonucleotide has been modified to improve the therapeutic or diagnostic properties of the oligonucleotide compound. In certain embodiments, at least one of the bases or at least one of the sugars of the oligonucleotide has been modified to improve the therapeutic or diagnostic properties of the oligonucleotide. The two strands of the oligonucleotide compound are complementary or partially complementary. Either strand or both strands may comprise a chimeric oligonucleotide. In certain embodiments, the double-stranded oligonucleotide is a siRNA. Another aspect of the present invention relates to a single-stranded oligonucleotide comprising at least one metal-chelating ligand. In certain embodiments, the oligonucleotide comprises at least one modified sugar moiety. In certain embodiments, the phosphate linkage in the oligonucleotide has been replaced with a phosphorothioate linkage.

In instances when the oligonucleotide is a siRNA, the oligonucleotide should include a region of sufficient homology to the target gene, and is of sufficient length in terms of nucleotides, such that the siRNA agent, or a fragment thereof, can mediate down-regulation of the target gene. It will be understood that the term “ribonucleotide” or “nucleotide” can, in the case of a modified RNA or nucleotide surrogate, also refer to a modified nucleotide, or surrogate replacement moiety at one or more positions. Thus, the siRNA agent is or includes a region which is at least partially complementary to the target RNA. In certain embodiments, the siRNA agent is fully complementary to the target RNA. It is not necessary that there be complete complementarity between the siRNA agent and the target, but the correspondence must be sufficient to enable the siRNA agent, or a cleavage product thereof, to direct sequence specific silencing, such as by RNAi cleavage of the target RNA. Complementarity, or the degree of homology with the target strand, is most critical in the antisense strand. While perfect complementarity, particularly in the antisense strand, is often desired some embodiments can include one or more but preferably 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule.

In addition, a siRNA agent will often be modified or include nucleoside surrogates. Single stranded regions of an siRNA agent will often be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA agent, e.g., against exonucleases, or to favor the antisense siRNA agent to enter into RISC are also favored. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

siRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter siRNA agent” as used refers to an siRNA agent that is sufficiently short that it does not induce a deleterious interferon response in a human cell, e.g., it has a duplexed region of less than 60 but preferably less than 50, 40, or 30 nucleotide pairs. The siRNA agent, or a cleavage product thereof, can down regulate a target gene, e.g., by inducing RNAi with respect to a target RNA, preferably an endogenous or pathogen target RNA.

Each strand of a siRNA agent can be equal to or less than 30, 25, 24, 23, 22, 21, or 20 nucleotides in length. The strand is generally at least 19 nucleotides in length. For example, each strand can be between 21 and 25 nucleotides in length. Exemplary siRNA agents have a duplex region of 17, 18, 19, 29, 21, 22, 23, 24, or 25 nucleotide pairs, and one or more overhangs, preferably one or two 3′ overhangs, of 2-3 nucleotides.

In addition to homology to target RNA and the ability to down regulate a target gene, an siRNA agent will generally have one or more of the following properties:

(1) it will, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA;

(2) it will, despite modifications, even to a very large number, or all of the nucleosides, still have “RNA-like” properties, i.e., it will possess the overall structural, chemical and physical properties of an RNA molecule, even though not exclusively, or even partly, of ribonucleotide-based content. For example, an siRNA agent can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. Generally, it is preferred that a modified moiety at the 2′ sugar position will be able to enter into H-bonding which is more characteristic of the OH moiety of a ribonucleotide than the H moiety of a deoxyribonucleotide. An exemplary siRNA agent will: exhibit a C_{3′}-endo pucker in all, or at least 50, 75,80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, orl sugar which is not a C_{3′}-endo pucker structure.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents are generally antisense with regard to the target molecule. A single strand iRNA agent should be sufficiently long that it can enter the RISC and participate in RISC mediated cleavage of a target mRNA. A single strand iRNA agent is at least 14, and more preferably at least 15, 20, 25, 29, 35, 40, or 50 nucleotides in length. It is preferably less than 200, 100, or 60 nucleotides in length.

Hairpin iRNA agents will have a duplex region equal to or at least 17, 18, 19, 29, 21, 22, 23, 24, or 25 nucleotide pairs. The duplex region will generally be equal to or less than 200, 100, or 50, in length. Exemplary ranges for the duplex region are 15-30, 17 to 23, 19 to 23, and 19 to 21 nucleotides pairs in length. The hairpin will generally have a single strand overhang or terminal unpaired region, often the 3′, and of the antisense side of the hairpin. Certain overhangs are 2-3 nucleotides in length.

Chimeric oligonucleotides, or “chimeras,” are oligonucleotides which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. Consequently, comparable results can often be obtained with shorter oligonucleotides when chimeric oligonucleotides are used. Chimeric oligonucleotides of the invention may be formed as composite structures of two or more oligonucleotides, modified oligonucleotides, oligonucleotides and/or oligonucleotide mimetics as described above. Such oligonucleotides have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; and 5,955,589, each of which is hereby incorporated by reference. In certain embodiments, the chimeric oligonucleotide is RNA-DNA, DNA-RNA, RNA-DNA-RNA, DNA-RNA-DNA, or RNA-DNA-RNA-DNA, wherein the oligonucleotide is between 5 and 60 nucleotides in length.

Certain compounds of the invention are described below in greater detail. Importantly, the embodiments described below are included merely for purposes of illustration of certain aspects and embodiments of the present invention, and are not intended to limit the invention.

Another aspect of the present invention relates to an oligonucleotide, or a pharmaceutically-acceptable salt thereof, comprising at least one strand represented independently by formula (I):

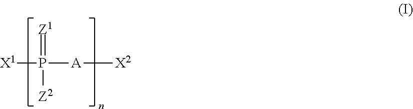

wherein, independently for each occurrence,

A is

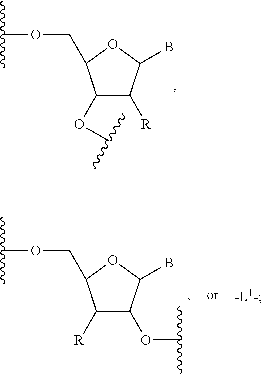

B is

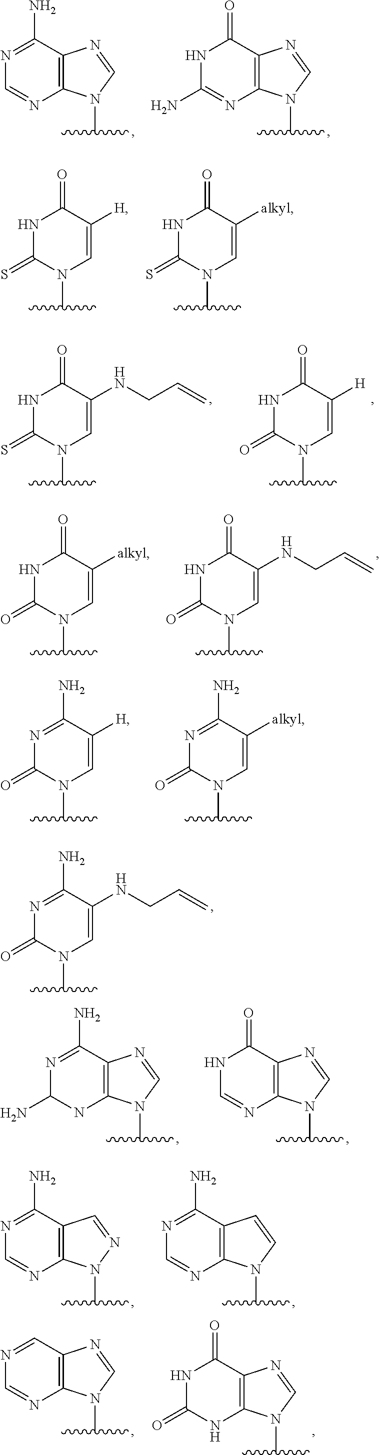

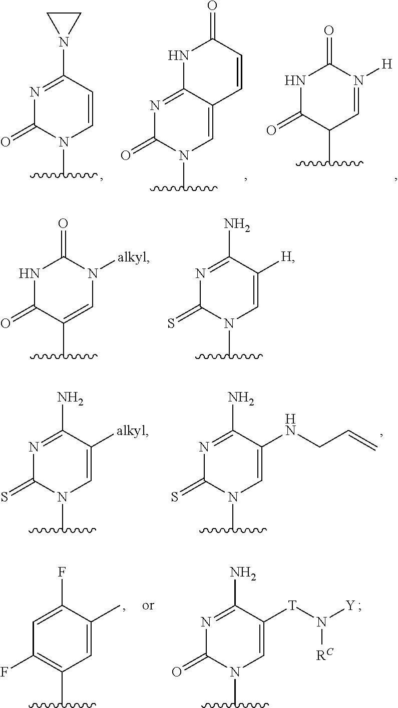

X¹ is —OR^{A},

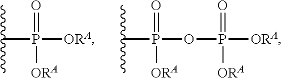

### or -L²;

X² is —H, —R^{B},

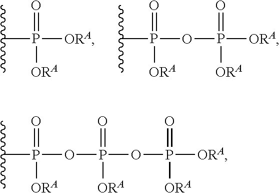

### or -L²;

Z¹ is O or S;

Z² is —OH, —OM¹, —O-alkyl, —O-aryl, —O-aralkyl, —SH, —SM¹, —S-alkyl, —S-aryl, —S-aralkyl, —N(R^{E})₂, —(C(R^{E})₂)ₘ—N(R^{E})₂, —N(R^{E})—(C(R^{E})₂)ₘ—N(R^{E})₂, or alkyl;

L¹ is

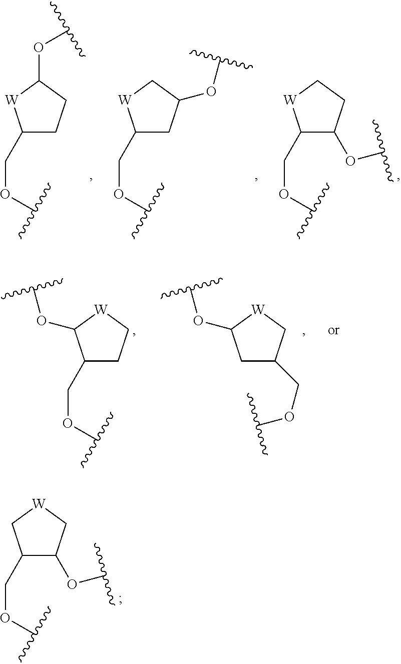

L² is

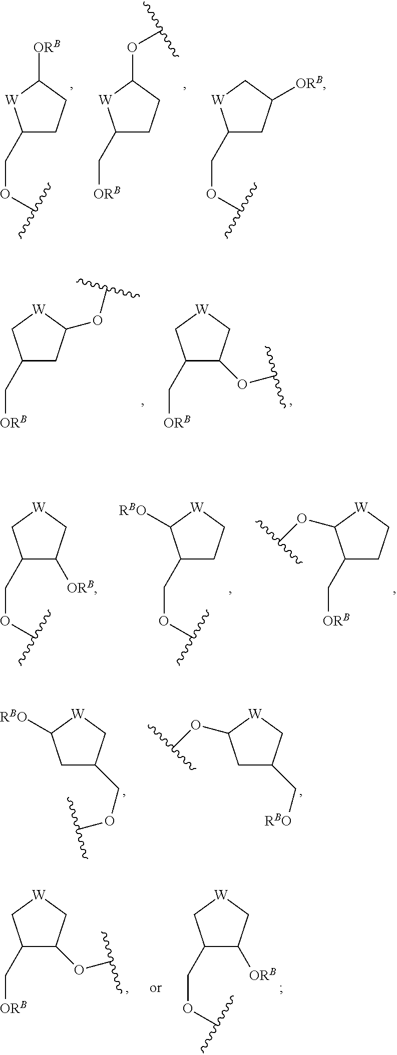

W is NC(═O)-T-N(R^{C})Y, or N-T-N(R^{C})Y;

T is —R^{D}—, —R^{D}—N(R^{C})—R^{D}— or —R^{D}—N(R^{C})C(═O)—R^{D}—;

Y is —H or —Z;

Z is a metal-chelating ligand or a metal-chelating ligand bound to M²;

R is —H, —OH, —F, —Cl, —Br, —O-alkyl, —O-allyl, —O—(C(R^{E})₂)ₘ—OR^{E}, —O—(C(R^{E})₂)ₘ—SR^{E}, —O—(C(R^{E})₂)ₘ—N(R^{E})₂, —O—(C(R^{E}₂)ₘ—C(O)N(R^{E})₂, —N(R^{E})₂, —S(C₁-C₆)alkyl, —O—(C(R^{E})₂)ₘ—O—(C₁-C₆)alkyl, —O—(C(R^{E})₂)ₘ—S(C₁-C₆)alkyl, —O(C(R^{E})₂)ₘ—O—(C(R^{E})₂)ₘ—N((C₁-C₆)alkyl)₂, or —O—(C(R^{E})₂)ₘ—O—N((C₁-C₆)alkyl)₂;

R^{A} is hydrogen, M¹ or alkyl;

R^{B} is hydrogen, alkyl, aryl, aralkyl, acyl or silyl;

R^{C} is hydrogen, alkyl, acyl, or aralkyl;

R^{D} is alkylene, heteroalkylene, alkenylene, alkynylene, arylene, or aralkylene;

R^{E} is hydrogen or alkyl; or two R^{E} taken together with the atoms to which they are bound form a 3-, 4-, 5-, 6-, or 7-membered carbocyclic or heterocyclic ring;

n is 0-200 inclusive;

m is 1-4 inclusive;

M¹ comprises an alkali metal ion or a transition metal ion with an overall charge of +1; and

M² is a paramagnetic metal ion or a lanthanide metal ion.

In certain embodiments, the present invention relates to any one of the aforementioned oligonucleotides, wherein n is 0-100 inclusive.

In certain embodiments, the present invention relates to any one of the aforementioned oligonucleotides, wherein n is 0-50 inclusive.

In certain embodiments, the present invention relates to any one of the aforementioned oligonucleotides, wherein n is 0-25 inclusive.

In certain embodiments, the present invention relates to any one of the aforementioned oligonucleotides, wherein n is 10-25 inclusive.

In certain embodiments, the present invention relates to any one of the aforementioned oligonucleotides, wherein n is 17-23 inclusive.

In certain embodiments, the present invention relates to any one of the aforementioned oligonucleotides, wherein n is 20.

In certain embodiments, the present invention relates to any one of the aforementioned oligonucleotides, wherein n is 20, and the first strand and the second strand are hybridized so that there are two un-hybridized nucleotides on the first strand and the second strand.

In certain embodiments, the present invention relates to any one of the aforementioned oligonucleotides, wherein n is 20 for the first strand; and n is 22 for the second strand.

In certain embodiments, the present invention relates to any one of the aforementioned oligonucleotides, wherein n is 19, and the first strand and the second strand are hybridized so that there are two un-hybridized nucleotides on the first strand and the second strand.

In certain embodiments, the present invention relates to any one of the aforementioned oligonucleotides, wherein n is 19 for the first strand; and n is 21 for the second strand.

In certain embodiments, the present invention relates to any one of the aforementioned oligonucleotides, wherein Z occurs only once. In certain embodiments, the present invention relates to any one of the aforementioned oligonucleotides, wherein Z occurs only twice.

In certain embodiments, the present invention relates to any one of the aforementioned oligonucleotides, wherein Z occurs at least one on the first strand.

In certain embodiments, the present invention relates to any one of the aforementioned oligonucleotides, wherein Z occurs at least one on the second strand.

In certain embodiments, the present invention relates to any one of the aforementioned oligonucleotides, wherein Z occurs at least one on the first strand; and Z does not occur on the second strand.

In certain embodiments, the present invention relates to any one of the aforementioned oligonucleotides, wherein A is

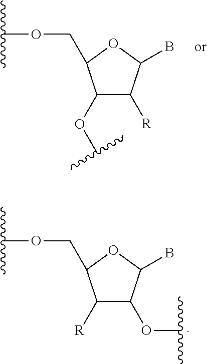

In certain embodiments, the present invention relates to any one of the aforementioned oligonucleotides, wherein A is

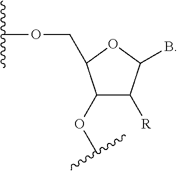

In certain embodiments, the present invention relates to any one of the aforementioned oligonucleotides, wherein A is

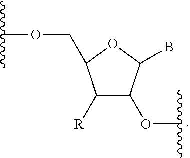

In certain embodiments, the present invention relates to any one of the aforementioned oligonucleotides, wherein B is

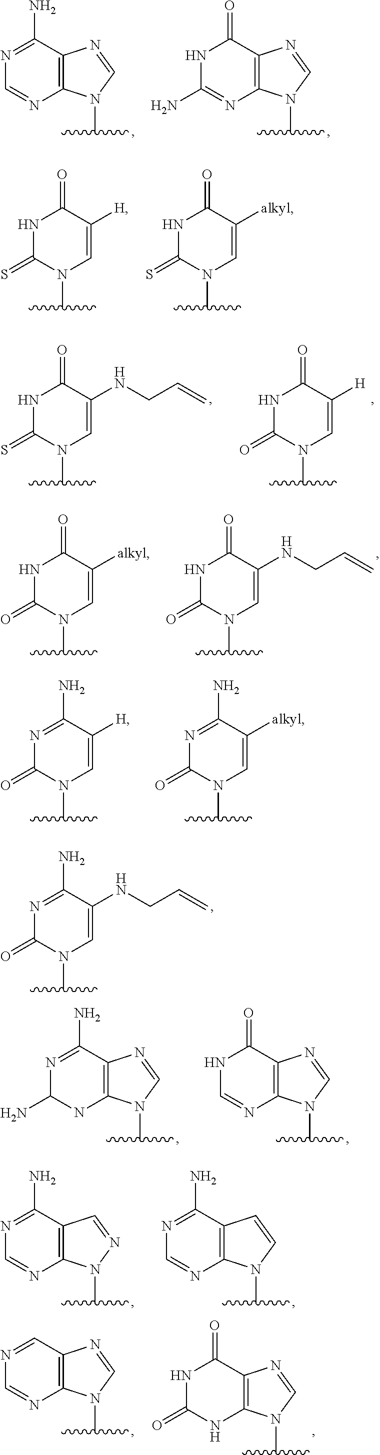

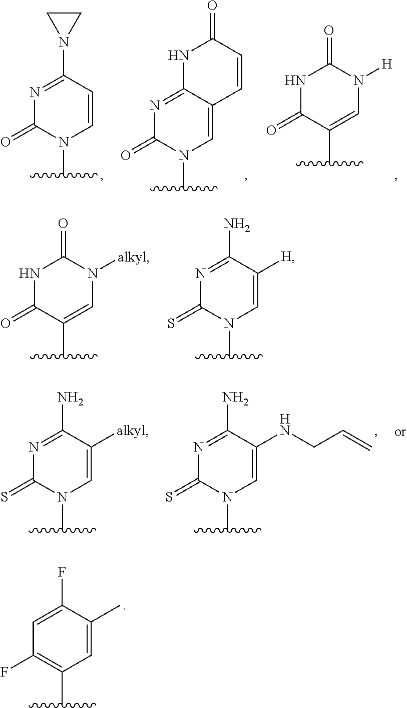

In certain embodiments, the present invention relates to any one of the aforementioned oligonucleotides, wherein B is

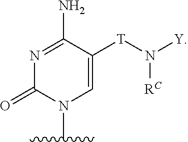

In certain embodiments, the present invention relates to any one of the aforementioned oligonucleotides, wherein X¹ is —OR^{A}.

In certain embodiments, the present invention relates to any one of the aforementioned oligonucleotides, wherein X¹ is -L².

In certain embodiments, the present invention relates to any one of the aforementioned oligonucleotides, wherein X² is —H or —R^{B}.

In certain embodiments, the present invention relates to any one of the aforementioned oligonucleotides, wherein X² is —H.

In certain embodiments, the present invention relates to any one of the aforementioned oligonucleotides, wherein Z¹ is O.

In certain embodiments, the present invention relates to any one of the aforementioned oligonucleotides, wherein Z¹ is S.

In certain embodiments, the present invention relates to any one of the aforementioned oligonucleotides, wherein Z² is —OH or —OM¹.

In certain embodiments, the present invention relates to any one of the aforementioned oligonucleotides, wherein Z² is —SH or —SM¹.

In certain embodiments, the present invention relates to any one of the aforementioned oligonucleotides, wherein L¹ is,

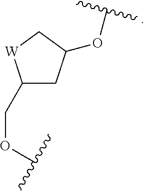

In certain embodiments, the present invention relates to any one of the aforementioned oligonucleotides, wherein L¹ is,

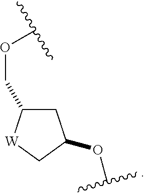

In certain embodiments, the present invention relates to any one of the aforementioned oligonucleotides, wherein L² is,

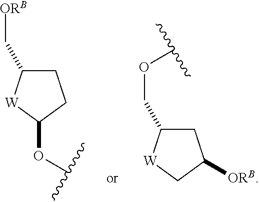

In certain embodiments, the present invention relates to any one of the aforementioned oligonucleotides, wherein W is NC(═O)-T-N(R^{C})Y.

In certain embodiments, the present invention relates to any one of the aforementioned oligonucleotides, wherein W is NC(═O)-T-N(H)Y.

In certain embodiments, the present invention relates to any one of the aforementioned oligonucleotides, wherein W is NC(═O)-T-N(H)Z.

In certain embodiments, the present invention relates to any one of the aforementioned oligonucleotides, wherein W is NC(═O)-T-NH₂.

In certain embodiments, the present invention relates to any one of the aforementioned oligonucleotides, wherein W is NC(═O)—R^{D}—N(H)Z.

In certain embodiments, the present invention relates to any one of the aforementioned oligonucleotides, wherein W is NC(═O)—R^{D}—N(H)C(═O)—R^{D}—N(H)Z.

In certain embodiments, the present invention relates to any one of the aforementioned oligonucleotides, wherein W is N-T-N(R^{C})Y.

In certain embodiments, the present invention relates to any one of the aforementioned oligonucleotides, wherein W is N-T-N(H)Y.

In certain embodiments, the present invention relates to any one of the aforementioned oligonucleotides, wherein W is N-T-N(H)Z.

In certain embodiments, the present invention relates to any one of the aforementioned oligonucleotides, wherein W is N-T-NH₂.

In certain embodiments, the present invention relates to any one of the aforementioned oligonucleotides, wherein W is N—R^{D}—N(H)Z.

In certain embodiments, the present invention relates to any one of the aforementioned oligonucleotides, wherein W is N—R^{D}—N(H)C(═O)—R^{D}—N(H)Z.

In certain embodiments, the present invention relates to any one of the aforementioned oligonucleotides, wherein T is —R^{D}—.

In certain embodiments, the present invention relates to any one of the aforementioned oligonucleotides, wherein T is —R^{D}—N(R^{C})—R^{D}—.

In certain embodiments, the present invention relates to any one of the aforementioned oligonucleotides, wherein T is —R^{D}—N(R^{C})C(═O)—R^{D}—.

In certain embodiments, the present invention relates to any one of the aforementioned oligonucleotides, wherein Y is H.

In certain embodiments, the present invention relates to any one of the aforementioned oligonucleotides, wherein at least one Y is Z.

In certain embodiments, the present invention relates to any one of the aforementioned oligonucleotides, wherein Z is a metal-chelating ligand.

In certain embodiments, the present invention relates to any one of the aforementioned oligonucleotides, wherein Z is a metal-chelating ligand bound to M².

In certain embodiments, the present invention relates to any one of the aforementioned oligonucleotides, wherein Z is a metal-chelating ligand; and Z is a MRI contrast agent.

In certain embodiments, the present invention relates to any one of the aforementioned oligonucleotides, wherein Z is a metal-chelating ligand bound to M²; and Z is a MRI contrast agent.

In certain embodiments, the present invention relates to any one of the aforementioned oligonucleotides, wherein Z is a metal-chelating ligand of formula (II) or (III):

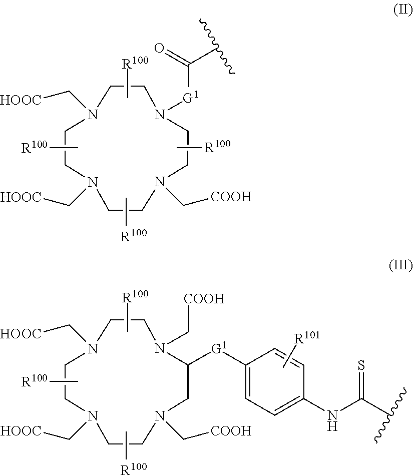

wherein, independently for each occurrence,

G¹ is alkylene, heteroalkylene, alkenylene, alkynylene, arylene, or aralkylene;

R¹⁰⁰ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In certain embodiments, the present invention relates to the aforementioned compound of formula (II) or (III), wherein G¹ is C₁-C₆ alkylene.

In certain embodiments, the present invention relates to the aforementioned compound of formula (II) or (III), wherein G¹ is —CH₂—, —CH₂CH₂— or —CH₂CH₂CH₂—.

In certain embodiments, the present invention relates to the aforementioned compound of formula (II) or (III), wherein G¹ is —CH₂—.

In certain embodiments, the present invention relates to the aforementioned compound of formula (II) or (III), wherein R¹⁰⁰ is hydrogen, halogen, hydroxy, alkyl, amino, sulfhydryl, alkylthio, alkoxy, or fluoroalkyl.

In certain embodiments, the present invention relates to the aforementioned compound of formula (II) or (III), wherein R¹⁰⁰ is hydrogen.

In certain embodiments, the present invention relates to the aforementioned compound of formula (II) or (III), wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (II) or (III), wherein R¹⁰¹ is hydrogen.

In certain embodiments, the present invention relates to any one of the aforementioned oligonucleotides, wherein Z is a metal-chelating ligand of formula (II) or (III) bound to M²:

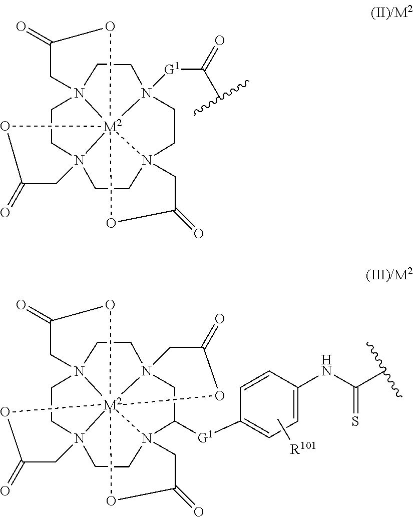

wherein, independently for each occurrence,

G¹ is alkylene, heteroalkylene, alkenylene, alkynylene, arylene, or aralkylene; and

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In certain embodiments, the present invention relates to the aforementioned compound of formula (II)/M² or (III)/M², wherein G¹ is C₁-C₆ alkylene.

In certain embodiments, the present invention relates to the aforementioned compound of formula (II)/M² or (III)/M², wherein G¹ is —CH₂—, —CH₂CH₂— or —CH₂₋CH₂CH₂-.

In certain embodiments, the present invention relates to the aforementioned compound of formula (II)/M² or (III)/M², wherein G¹ is —CH₂—.

In certain embodiments, the present invention relates to the aforementioned compound of formula (II)/M² or (III)/M², wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (II)/M² or (III)/M², wherein R¹⁰¹ is hydrogen.

In certain embodiments, the present invention relates to any one of the aforementioned oligonucleotides, wherein Z is a metal-chelating ligand of formula (IV):

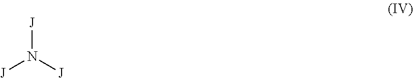

wherein, independently for each occurrence,

J is

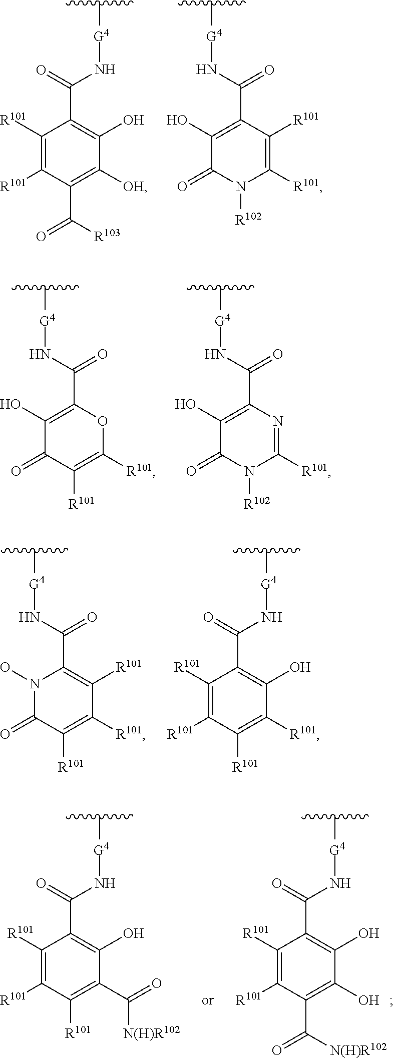

G⁴ is -T⁴- or -T⁴-N(H)C(═O)-T⁴-;

T⁴ is alkylene, heteroalkylene, alkenylene, alkynylene, arylene, or aralkylene;

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

R¹⁰² is hydrogen, alkyl, alkenyl, alkynyl, cycloalkyl, heterocyclyl, aryl, aralkyl, heteroaryl, heteroaralkyl, or fluoroalkyl.

In certain embodiments, the present invention relates to the aforementioned compound of formula (IV), wherein G⁴ is C₁-C₆ alkylene.

In certain embodiments, the present invention relates to the aforementioned compound of formula (IV), wherein G⁴ is —CH₂—, —CH₂CH₂— or —CH₂CH₂CH₂—.

In certain embodiments, the present invention relates to the aforementioned compound of formula (IV), wherein G⁴ is —CH₂CH₂—.

In certain embodiments, the present invention relates to the aforementioned compound of formula (IV), wherein G⁴ is —CH₂CH(CH₂OH)—.

In certain embodiments, the present invention relates to the aforementioned compound of formula (IV), wherein R¹⁰¹ is hydrogen, halogen, hydroxy, alkyl, amino, sulthydryl, alkylthio, alkoxy, or fluoroalkyl.

In certain embodiments, the present invention relates to the aforementioned compound of formula (IV), wherein R¹⁰¹ is hydrogen.

In certain embodiments, the present invention relates to the aforementioned compound of formula (IV), wherein R¹⁰² is hydrogen, alkyl, or aralkyl.

In certain embodiments, the present invention relates to the aforementioned compound of formula (IV), wherein R¹⁰² is hydrogen.

In certain embodiments, the present invention relates to the aforementioned compound of formula (IV), wherein said compound is selected from the group consisting of

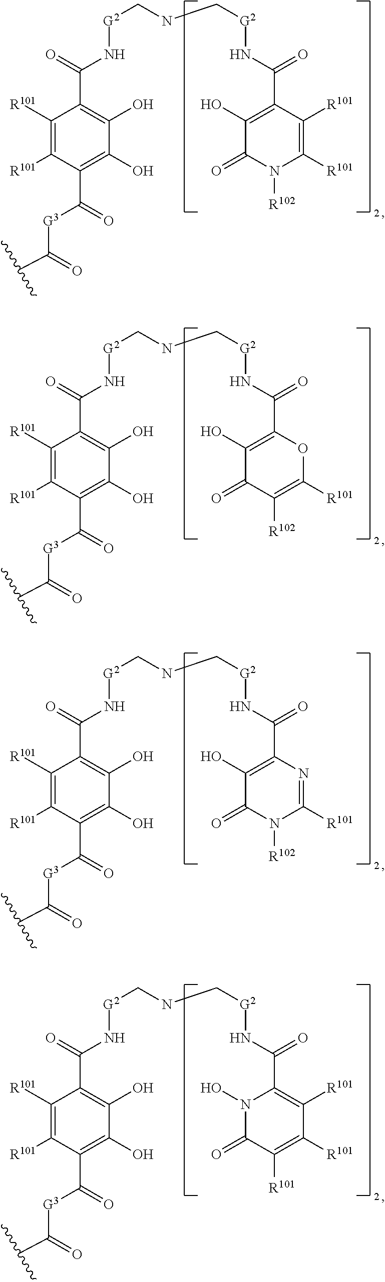

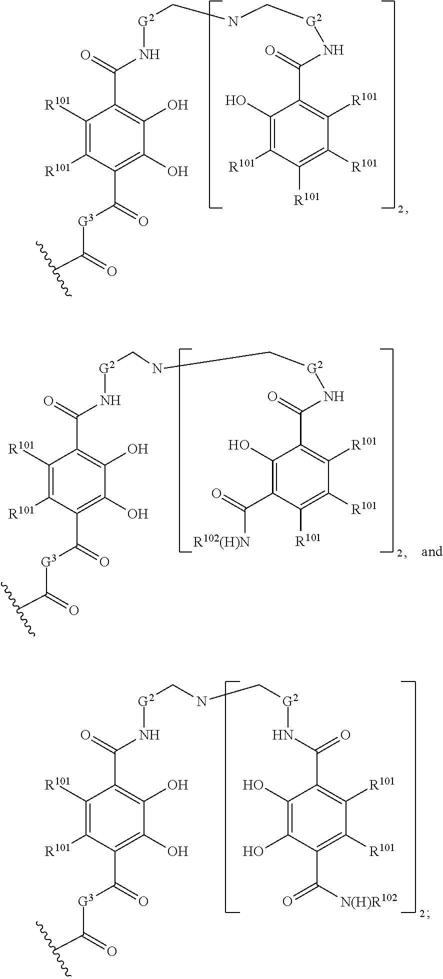

G² is alkylene; and
G³ is alkylene or heteroalkylene. Alternatively, G³ is alkylamine or dialkylamine, wherein the alkyl moiety is optionally substituted with halogen, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In certain embodiments, the present invention relates to the aforementioned compound of formula (IV), wherein G² is —CH₂—, —CH₂CH₂— or —CH₂CH₂CH₂—.

In certain embodiments, the present invention relates to the aforementioned compound of formula (IV), wherein G² is —CH₂—.

In certain embodiments, the present invention relates to the aforementioned compound of formula (IV), wherein G³ is alkylene.

In certain embodiments, the present invention relates to the aforementioned compound of formula (IV), wherein G³ is —(CH₂)₅—.

In certain embodiments, the present invention relates to any one of the aforementioned oligonucleotides, wherein Z is a metal-chelating ligand of formula (IV) bound to M²:

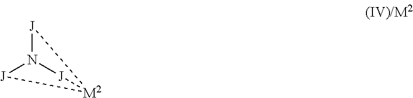

wherein, independently for each occurrence,

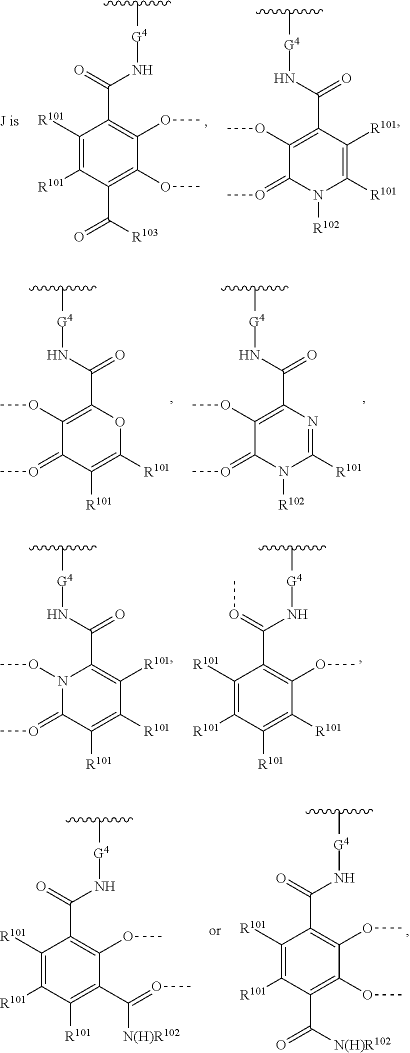

G⁴ is -T⁴- or -T⁴-N(H)C(═O)-T⁴-;

T⁴ is alkylene, heteroalkylene, alkenylene, alkynylene, arylene, or aralkylene;

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

R¹⁰² is hydrogen, alkyl, alkenyl, alkynyl, cycloalkyl, heterocyclyl, aryl, aralkyl, heteroaryl, heteroaralkyl, or fluoroalkyl.

In certain embodiments, the present invention relates to the aforementioned compound of formula (IV)/M², wherein G⁴ is C₁-C₆ alkylene.

In certain embodiments, the present invention relates to the aforementioned compound of formula (IV)/M², wherein G⁴ is —CH₂—, —CH₂CH₂— or —CH₂CH₂CH₂—.

In certain embodiments, the present invention relates to the aforementioned compound of formula (IV)/M², wherein G⁴ is —CH₂CH₂—.

In certain embodiments, the present invention relates to the aforementioned compound of formula (IV)/M², wherein G⁴ is —CH₂CH(CH₂OH)—.

In certain embodiments, the present invention relates to the aforementioned compound of formula (IV)/M², wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (IV)/M², wherein R¹⁰¹ is hydrogen.

In certain embodiments, the present invention relates to the aforementioned compound of formula (IV)/M², wherein R¹⁰² is hydrogen, alkyl, or aralkyl.

In certain embodiments, the present invention relates to the aforementioned compound of formula (IV)/M², wherein R¹⁰² is hydrogen.

In certain embodiments, the present invention relates to the aforementioned compound of formula (IV) bound to M², wherein said compound is selected from the group consisting of

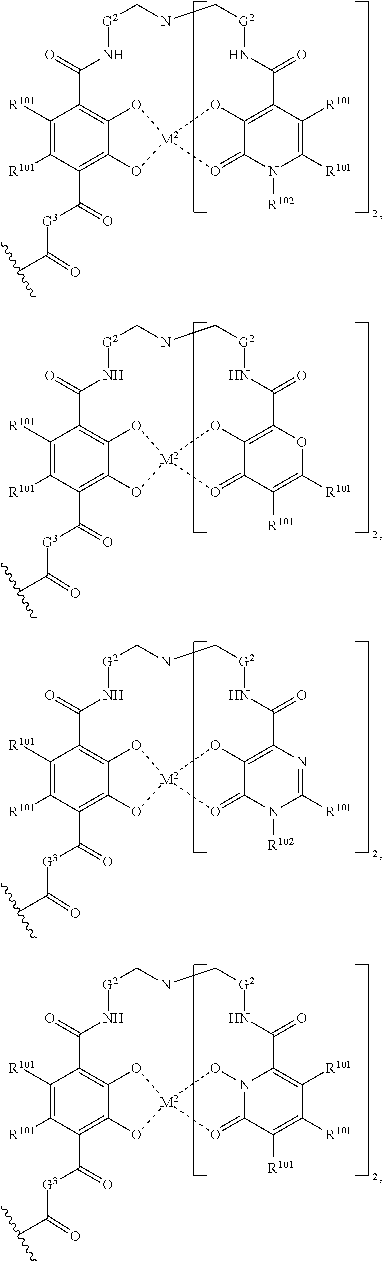

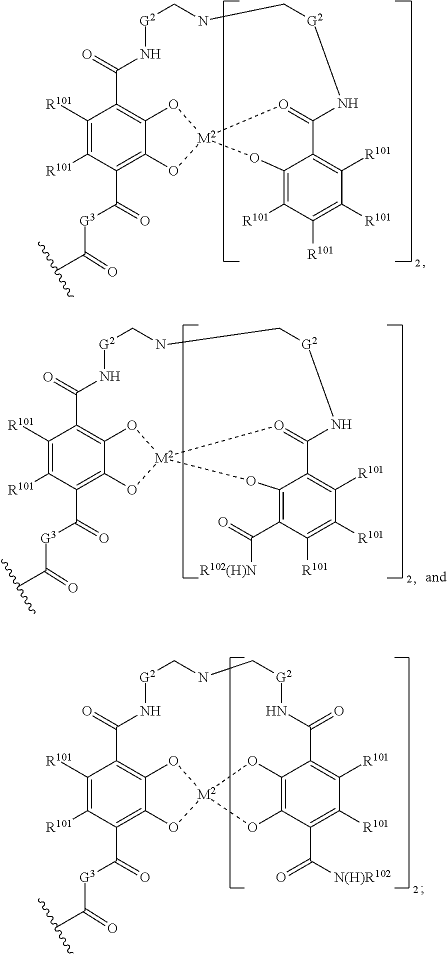

G² is alkylene; and
G³ is alkylene or heteroalkylene. Alternatively, G³ is alkylamine or dialkylamine, wherein the alkyl moiety is optionally substituted with halogen, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In certain embodiments, the present invention relates to the aforementioned compound of formula (IV)/M², wherein G² is —CH₂—, —CH₂CH₂— or —CH₂CH₂CH₂—.

In certain embodiments, the present invention relates to the aforementioned compound of formula (IV)/M², wherein G² is —CH₂—.

In certain embodiments, the present invention relates to the aforementioned compound of formula (IV)/M², wherein G³ is alkylene.

In certain embodiments, the present invention relates to the aforementioned compound of formula (IV)/M², wherein G³ is —(CH₂)₅—.

In certain embodiments, the present invention relates to any one of the aforementioned oligonucleotides, wherein Z is a metal-chelating ligand of formula (V):

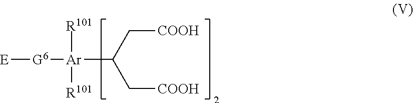

wherein, independently for each occurrence,

Ar is aryl or heteroaryl;

G⁶ is alkylene, heteroalkylene, alkenylene, alkynylene, arylene, or aralkylene;

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

E is —(C═O)— or -Ph-N—C(═S)—.

In certain embodiments, the present invention relates to the aforementioned compound of formula (V), wherein Ar is aryl.

In certain embodiments, the present invention relates to the aforementioned compound of formula (V), wherein Ar is phenyl.

In certain embodiments, the present invention relates to the aforementioned compound of formula (V), wherein Ar is heteroaryl.

In certain embodiments, the present invention relates to the aforementioned compound of formula (V), wherein Ar is pyridyl.

In certain embodiments, the present invention relates to the aforementioned compound of formula (V), wherein G⁶ is alkylene or heteroalkylene.

In certain embodiments, the present invention relates to the aforementioned compound of formula (V), wherein G⁶ is alkylene.

In certain embodiments, the present invention relates to the aforementioned compound of formula (V), wherein G⁶ is heteroalkylene.

In certain embodiments, the present invention relates to the aforementioned compound of formula (V), wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (V), wherein R¹⁰¹ is hydrogen.

In certain embodiments, the present invention relates to the aforementioned compound of formula (V), wherein E is —(C═O)—.

In certain embodiments, the present invention relates to the aforementioned compound of formula (V), wherein E is -Ph-N—C(═S)—.

In certain embodiments, the present invention relates to the aforementioned compound of formula (V), wherein said compound is

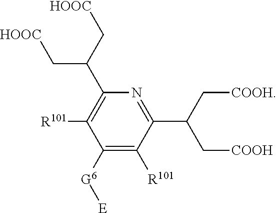

In certain embodiments, the present invention relates to any one of the aforementioned oligonucleotides, wherein Z is a metal-chelating ligand of formula (V) bound to M²:

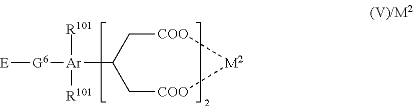

wherein, independently for each occurrence,

Ar is aryl or heteroaryl;

G⁶ is alkylene, heteroalkylene, alkenylene, alkynylene, arylene, or aralkylene;

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

E is —(C═O)— or -Ph-N—C(═S)—.

In certain embodiments, the present invention relates to the aforementioned compound of formula (V)/M², wherein Ar is aryl.

In certain embodiments, the present invention relates to the aforementioned compound of formula (V)/M², wherein Ar is phenyl.

In certain embodiments, the present invention relates to the aforementioned compound of formula (V)/M², wherein Ar is heteroaryl.

In certain embodiments, the present invention relates to the aforementioned compound of formula (V)/M², wherein Ar is pyridyl.

In certain embodiments, the present invention relates to the aforementioned compound of formula (V)/M², wherein G⁶ is alkylene or heteroalkylene.

In certain embodiments, the present invention relates to the aforementioned compound of formula (V)/M², wherein G⁶ is alkylene.

In certain embodiments, the present invention relates to the aforementioned compound of formula (V)/M², wherein G⁶ is heteroalkylene.

In certain embodiments, the present invention relates to the aforementioned compound of formula (V)/M², wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (V)/M², wherein R¹⁰¹ is hydrogen.

In certain embodiments, the present invention relates to the aforementioned compound of formula (V)/M², wherein E is —(C═O)—.

In certain embodiments, the present invention relates to the aforementioned compound of formula (V)/M², wherein E is -Ph-N—C(═S)—.

In certain embodiments, the present invention relates to the aforementioned compound of formula (V)/M², wherein said compound is

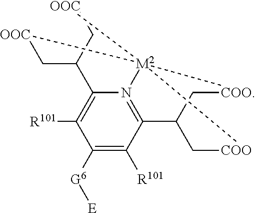

In certain embodiments, the present invention relates to any one of the aforementioned oligonucleotides, wherein Z is a metal-chelating ligand of formula (VI), (VII) or (VIII):

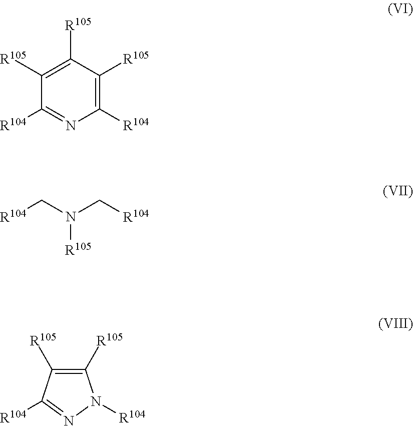

wherein, independently for each occurrence,

R¹⁰⁴ is

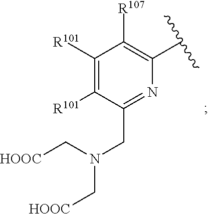

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;

R¹⁰⁷ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;

R¹⁰⁵ is -alkylene-E, -heteroalkylene-E, -alkenylene-E, -alkynylene-E, -arylene-E, or -aralkylene-E; and

E is —(C═O)— or -Ph-N—C(═S)—.

In certain embodiments, the present invention relates to the aforementioned compound of formula (VI), (VII) or (VIII), wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (VI), (VII) or (VIII), wherein R¹⁰¹ is hydrogen.

In certain embodiments, the present invention relates to the aforementioned compound of formula (VI), (VII) or (VIII), wherein R¹⁰⁷ is hydrogen, halogen, hydroxy, alkyl, amino, sulfhydryl, alkylthio, alkoxy, or fluoroalkyl.

In certain embodiments, the present invention relates to the aforementioned compound of formula (VI), (VII) or (VIII), wherein R¹⁰⁷ is aryl or heteroaryl.

In certain embodiments, the present invention relates to the aforementioned compound of formula (VI), (VII) or (VIII), wherein R¹⁰⁷ is aryl.

In certain embodiments, the present invention relates to the aforementioned compound of formula (VI), (VII) or (VIII), wherein R¹⁰⁷ is heteroaryl.

In certain embodiments, the present invention relates to the aforementioned compound of formula (VI), (VII) or (VIII), wherein R¹⁰⁵ is -alkylene-E or -heteroalkylene-E.

In certain embodiments, the present invention relates to the aforementioned compound of formula (VI), (VII) or (VIII), wherein R¹⁰⁵ is -alkylene-E.

In certain embodiments, the present invention relates to the aforementioned compound of formula (VI), (VII) or (VIII), wherein R¹⁰⁵ is -heteroalkylene-E.

In certain embodiments, the present invention relates to the aforementioned compound of formula (VI), (VII) or (VIII), wherein E is —(C═O)—.

In certain embodiments, the present invention relates to the aforementioned compound of formula (VI), (VII) or (VIII), wherein E is -Ph-N—C(═S)—.

In certain embodiments, the present invention relates to the aforementioned compound of formula (VI), (VII) or (VIII), wherein said compound is selected from the group consisting of

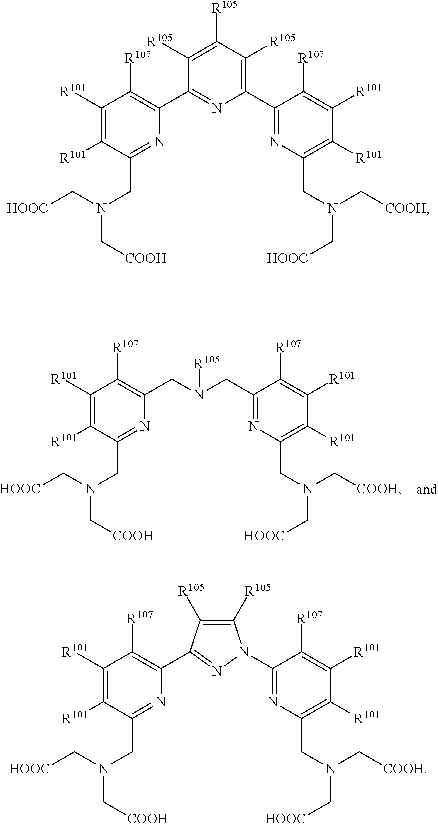

In certain embodiments, the present invention relates to any one of the aforementioned oligonucleotides, wherein Z is a metal-chelating ligand of formula (VI), (VII) or (VIII) bound to M²:

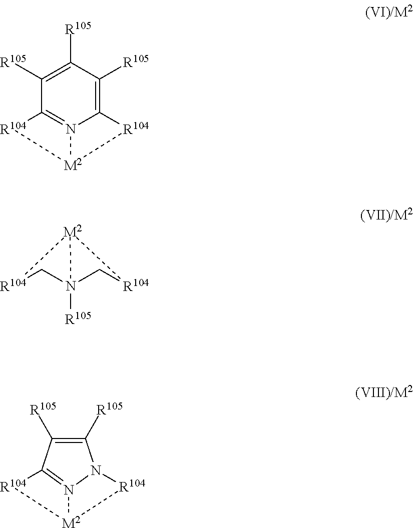

wherein, independently for each occurrence,

R¹⁰⁴ is

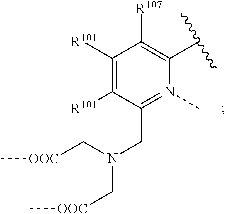

R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;

R¹⁰⁷ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;

R¹⁰⁵ is -alkylene-E, -heteroalkylene-E, -alkenylene-E, -alkynylene-E, -arylene-E, or -aralkylene-E; and

E is —(C═O)— or -Ph-N—C(═S)—.

In certain embodiments, the present invention relates to the aforementioned compound of formula (VI)/M², (VII)/M² or (VIII)/M², wherein R¹⁰¹ is hydrogen, halogen, hydroxy, alkyl, amino, sulfhydryl, alkylthio, alkoxy, or fluoroalkyl.

In certain embodiments, the present invention relates to the aforementioned compound of formula (VI)/M², (VII)/M² or (VIII)/M², wherein R¹⁰¹ is hydrogen.

In certain embodiments, the present invention relates to the aforementioned compound of formula (VI)/M², (VII)/M² or (VIII)/M², wherein R¹⁰⁷ is hydrogen, halogen, hydroxy, alkyl, amino, sulfhydryl, alkylthio, alkoxy, or fluoroalkyl.

In certain embodiments, the present invention relates to the aforementioned compound of formula (VI)/M², (VII)/M² or (VIII)/M², wherein R¹⁰⁷ is aryl or heteroaryl.

In certain embodiments, the present invention relates to the aforementioned compound of formula (VI)/M², (VII)/M² or (VIII)/M², wherein R¹⁰⁷ is aryl.

In certain embodiments, the present invention relates to the aforementioned compound of formula (VI)/M², (VII)/M² or (VIII)/M², wherein R¹⁰⁷ is heteroaryl.

In certain embodiments, the present invention relates to the aforementioned compound of formula (VI)/M², (VII)/M² or (VIII)/M², wherein R¹⁰⁵ is -alkylene-E or -heteroalkylene-E.

In certain embodiments, the present invention relates to the aforementioned compound of formula (VI)/M², (VII)/M² or (VIII)/M², wherein R¹⁰⁵ is -alkylene-E.

In certain embodiments, the present invention relates to the aforementioned compound of formula (VI)/M², (VII)/M² or (VIII)/M², wherein R¹⁰⁵ is -heteroalkylene-E.

In certain embodiments, the present invention relates to the aforementioned compound of formula (VI)/M², (VII)/M² or (VIII)/M², wherein E is —(C═O)—.

In certain embodiments, the present invention relates to the aforementioned compound of formula (VI)/M², (VII)/M² or (VIII)/M², wherein E is -Ph-N—C(═S)—.

In certain embodiments, the present invention relates to the aforementioned compound of formula (VI)84², (VII)/M² or (VIII)/M², wherein said compound is selected

from the group consisting of

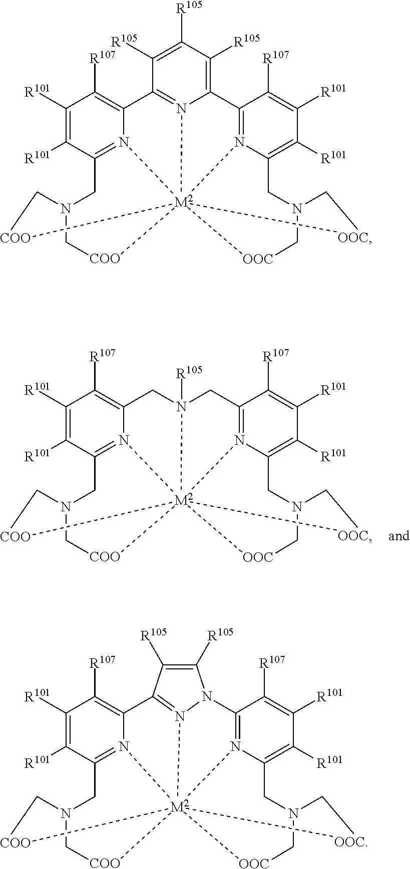

In certain embodiments, the present invention relates to any one of the aforementioned oligonucleotides, wherein Z is a metal-chelating ligand; and Z comprises an organic chromophore.

In certain embodiments, the present invention relates to any one of the aforementioned oligonucleotides, wherein Z is a metal-chelating ligand bound to M²; and Z comprises an organic chromophore.

In certain embodiments, the present invention relates to any one of the aforementioned oligonucleotides, wherein Z is a metal-chelating ligand of formula (IX):

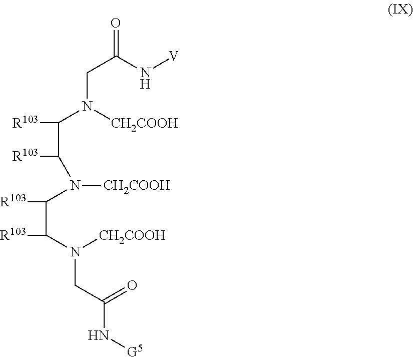

wherein, independently for each occurrence,

V is an organic chromophore; for example, organic chromophore can be one or more of the following types of compounds: azo, diazo, disazo, trisazo, diphenylmethane, triphenylmethane, xanthene, nitro, nitroso, acridine, methine, styryl, indamine, thiazole, oxazine, stilbene, or anthraquinone. In an alternative embodiment, the chromophore may be optically inactive, at least within the visible spectrum, but absorb uv radiation;

G⁵ is -alkylene-E, -heteroalkylene-E, -alkenylene-E, -alkynylene-E, -arylene-E, or -aralkylene-E;

R¹⁰³ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

E is —(C═O)— or -Ph-N—C(═S)—.

In certain embodiments, the present invention relates to the aforementioned compound of formula (IX), wherein V is aryl, heteroaryl, aralkyl, heteroaralkyl or polycyclyl.

In certain embodiments, the present invention relates to the aforementioned compound of formula (IX), wherein V is aralkyl or heteroaralkyl.

In certain embodiments, the present invention relates to the aforementioned compound of formula (IX), wherein V is polycyclyl.

In certain embodiments, the present invention relates to the aforementioned compound of formula (IX), wherein R¹⁰³ is hydrogen, halogen, hydroxy, alkyl, amino, sulfhydryl, alkylthio, alkoxy, or fluoroalkyl.

In certain embodiments, the present invention relates to the aforementioned compound of formula (IX), wherein R¹⁰³ is hydrogen.

In certain embodiments, the present invention relates to the aforementioned compound of formula (IX), wherein R¹⁰⁵ is -alkylene-E or -heteroalkylene-E.

In certain embodiments, the present invention relates to the aforementioned compound of formula (IX), wherein R¹⁰⁵ is -alkylene-E.

In certain embodiments, the present invention relates to the aforementioned compound of formula (IX), wherein R¹⁰⁵ is -heteroalkylene-E.

In certain embodiments, the present invention relates to the aforementioned compound of formula (IX), wherein E is —(C═O)—.

In certain embodiments, the present invention relates to the aforementioned compound of formula (IX), wherein E is -Ph-N—C(═S)—.

In certain embodiments, the present invention relates to the aforementioned compound of formula (IX), wherein said compound is

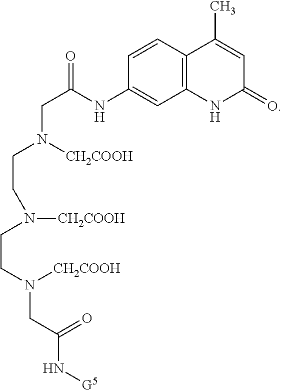

In certain embodiments, the present invention relates to any one of the aforementioned oligonucleotides, wherein Z is a metal-chelating ligand of formula (IX) bound to M²:

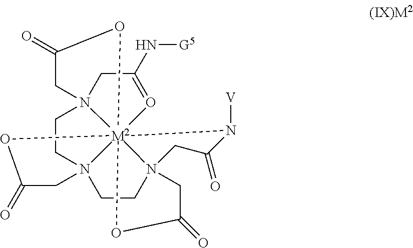

wherein, independently for each occurrence,

V is an organic chromophore; for example, organic chromophore can be one or more of the following types of compounds: azo, diazo, disazo, trisazo, diphenylmethane, triphenylmethane, xanthene, nitro, nitroso, acridine, methine, styryl, indamine, thiazole, oxazine, stilbene, or anthraquinone. In an alternative embodiment, the chromophore may be optically inactive, at least within the visible spectrum, but absorb uv radiation;

G⁵ is -alkylene-E, -heteroalkylene-E, -alkenylene-E, -alkynylene-E, -arylene-E, or -aralkylene-E;

R¹⁰³ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and

E is —(C═O)— or -Ph-N—C(═S)—.

In certain embodiments, the present invention relates to the aforementioned compound of formula (IX)/M², wherein V is aryl, heteroaryl, aralkyl, heteroaralkyl or polycyclyl.

In certain embodiments, the present invention relates to the aforementioned compound of formula (IX)/M², wherein V is aralkyl or heteroaralkyl.

In certain embodiments, the present invention relates to the aforementioned compound of formula (IX)/M², wherein V is polycyclyl.

In certain embodiments, the present invention relates to the aforementioned compound of formula (IX)/M², wherein R¹⁰³ is hydrogen, halogen, hydroxy, alkyl, amino, sulfhydryl, alkylthio, alkoxy, or fluoroalkyl.

In certain embodiments, the present invention relates to the aforementioned compound of formula (IX)/M², wherein R¹⁰³ is hydrogen.

In certain embodiments, the present invention relates to the aforementioned compound of formula (IX)/M², wherein R¹⁰⁵ is -alkylene-E or -heteroalkylene-E.

In certain embodiments, the present invention relates to the aforementioned compound of formula (IX)/M², wherein R¹⁰⁵ is -alkylene-E.

In certain embodiments, the present invention relates to the aforementioned compound of formula (IX)/M², wherein R¹⁰⁵ is -heteroalkylene-E.

In certain embodiments, the present invention relates to the aforementioned compound of formula (IX)/M², wherein E is —(C═O)—.

In certain embodiments, the present invention relates to the aforementioned compound of formula (IX)/M², wherein E is -Ph-N—C(═S)—.

In certain embodiments, the present invention relates to the aforementioned compound of formula (IX)/M², wherein said compound is

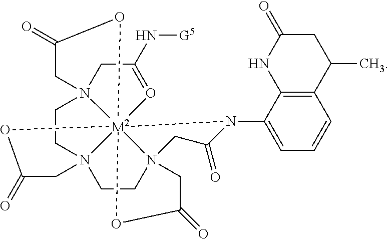

In certain embodiments, the present invention relates to any one of the aforementioned oligonucleotides, wherein R is H or OH

In certain embodiments, the present invention relates to any one of the aforementioned oligonucleotides, wherein R is H.

In certain embodiments, the present invention relates to any one of the aforementioned oligonucleotides, wherein R is OH.

In certain embodiments, the present invention relates to any one of the aforementioned oligonucleotides, wherein R^{A} is H.

In certain embodiments, the present invention relates to any one of the aforementioned oligonucleotides, wherein R^{B} is H or silyl.

In certain embodiments, the present invention relates to any one of the aforementioned oligonucleotides, wherein R^{C} is H.

In certain embodiments, the present invention relates to any one of the aforementioned oligonucleotides, wherein R^{D} is alkylene or heteroalkylene.

In certain embodiments, the present invention relates to any one of the aforementioned oligonucleotides, wherein R^{D} is alkylene.

In certain embodiments, the present invention relates to any one of the aforementioned oligonucleotides, wherein R^{E} is hydrogen.

In certain embodiments, the present invention relates to any one of the aforementioned oligonucleotides, wherein m is 1. In certain embodiments, the present invention relates to any one of the aforementioned oligonucleotides, wherein m is 2. In certain embodiments, the present invention relates to any one of the aforementioned oligonucleotides, wherein m is 3. In certain embodiments, the present invention relates to any one of the aforementioned oligonucleotides, wherein m is 4.

In certain embodiments, the present invention relates to any one of the aforementioned oligonucleotides, wherein M² is a paramagnetic metal ion.

In certain embodiments, the present invention relates to any one of the aforementioned oligonucleotides, wherein M² is a lanthanide metal ion.

In certain embodiments, the present invention relates to any one of the aforementioned oligonucleotides, wherein M² is Gd(III), Mn(II), Mn(III), Cr(II), Cr(III), Cu(II), Fe (III), Pr(III), Nd(III), Sm(III), Tb(III), Yb(III) Dy(III), Ho(III), Eu(II), Eu(III), or Er(III).

In certain embodiments, the present invention relates to any one of the aforementioned oligonucleotides, wherein M² is Gd(III) or Eu(III).

In certain embodiments, the present invention relates to any one of the aforementioned oligonucleotides, wherein M² is Gd(III).

In certain embodiments, the present invention relates to any one of the aforementioned oligonucleotides, wherein M² is Eu(III).

### Pharmaceutical Compositions

In certain aspects of the present disclosure is provided a composition comprising a compound of the invention, or a pharmaceutically acceptable salt thereof; and a pharmaceutically acceptable carrier.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, TRIS (tris(hydroxymethyl)amino-methane), partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle-ne-block polymers, polyethylene glycol and wool fat.

According to this disclosure, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

In some cases, depending on the dose and rate of injection, the binding sites on plasma proteins may become saturated with prodrug and activated agent. This leads to a decreased fraction of protein-bound agent and could compromise its half-life or tolerability as well as the effectiveness of the agent. In these circumstances, it is desirable to inject the prodrug agent in conjunction with a sterile albumin or plasma replacement solution. Alternatively, an apparatus/syringe can be used that contains the contrast agent and mixes it with blood drawn up into the syringe; this is then re-injected into the patient.

The compounds, diagnostic agents and pharmaceutical compositions of the present disclosure may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term “parenteral” as used herein includes subcutaneous, intravenous, intramuscular, intra-articular, intra-synovial, intrasternal, intrathecal, intrahepatic, intralesional and intracranial injection or infusion techniques.

When administered orally, the pharmaceutical compositions of this disclosure may be administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, when administered in the form of suppositories for rectal administration, the pharmaceutical compositions of this disclosure may be prepared by mixing the agent with a suitable non-irritating excipient that is solid at room temperature but liquid at rectal temperature and therefore will melt in the rectum to release the drug. Such materials include cocoa butter, beeswax and polyethylene glycols.

As noted before, the pharmaceutical compositions of this disclosure may also be administered topically, especially when the target of treatment includes areas or organs readily accessible by topical application, including the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds and/or diagnostic agents of this disclosure include, but are not limited to, mineral oil, liquid petrolatum, white petrolatum, propylene glycol, poly-oxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, typically, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

For administration by nasal aerosol or inhalation, the pharmaceutical compositions of this disclosure are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated and the particular mode of administration. A typical preparation will contain from about 5% to about 95% active compound (w/w). Typically, such preparations contain from about 20% to about 80% active compound.

For intravenous and other types of administration, acceptable dose ranges range from about 0.001 to about 1.0 mmol/kg of body weight, with the typical dose of the active ingredient compound ranging from about 0.001 to about 0.5 mmol/kg of body weight. Even more typical is from about 0.01 to about 0.1 mmol/kg, and the most typical dose of the active ingredient compound is from about 0.0001 and to about 0.05 mmol/kg.

As the skilled artisan will appreciate, lower or higher doses than those recited above may be required. Specific dosage regimens for any particular patient will depend upon a variety of factors, including the activity of the specific compound employed, the age, body weight, general health status, sex, diet, time of administration, rate of excretion, drug combination and the judgment of the treating physician.

As used herein, the phrase “pharmaceutically acceptable” refers to those compounds, diagnostic agents, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The compounds and/or diagnostic agents of the present disclosure can exist as pharmaceutically acceptable salts. The term “pharmaceutically acceptable salt,” as used herein, represents salts or zwitterionic forms of the compounds and/or diagnostic agents of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use The salts can be prepared during the final isolation and purification of the compounds and/or diagnostic agents or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds and/or diagnostic agents by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, meglumine, piperidine, and piperazine.

### Synthetic Methods

In general, the compounds of the present invention may be prepared by the methods illustrated in the general reaction schemes or by modifications thereof, using readily available starting materials, reagents and conventional synthesis procedures. In these reactions, it is also possible to make use of variants which are in themselves known, but are not mentioned here.

Certain compounds of the present invention may exist in particular geometric or stereoisomeric forms. Unless specified otherwise, the present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In certain instances, conjugation of ligand molecules is achieved by conjugation of the metal-chelating ligand to an amino tether on the nucleoside. This can be effected in several ways. For example, a metal-chelating ligand-nucleoside conjugate of the invention can be prepared by conjugation of the metal-chelated ligand molecule to the nucleoside using EDC/sulfo-NHS (i.e., 1-ethyl-3-(3-dimethylaminopropylcarbodiimide/N-hydroxysulfosuccinimide) to conjugate the carboxylate function of the ligand with the amino function of the linking group on the nucleoside. In a second step, a metal ion can be bound to the ligand. Alternatively, a metal-chelating ligand which is bound to a metal ion can be used.

In certain embodiments, the ligand-conjugated oligonucleotides of the present invention may be prepared by conjugation of the metal-chelating ligand molecule to the nucleoside sequence via a heterobifunctional tether such as m-maleimidobenzoyl-N-hydroxysulfosuccinimide ester (MBS) or succinimidyl 4-(N-maieimidomethyl)cyclo-hexane-1-carboxylate (SMCC), to link a nucleophilic position on the ligand molecule to the amino function of the tether group on nucleoside sequence. By this mechanism, an oligonucleoside-maleimide conjugate is formed by reaction of the amino group of the tether on the linked nucleosides with the MBS or SMCC maleimide linker. The conjugate is then reacted with the ligand.

In other embodiments, a metal-chelating ligand conjugated-oligonucleotide can be prepared by conjugation of the ligand molecule to the oligonucleotide or nucleoside via a homobifunctional tether such as disuccinimidyl suberate (DSS), to link an amino function on the ligand to the amino group of a tether on the oligonucleotide sequence. By this mechanism, an oligonucleoside-succinimidyl conjugate is formed by reaction of the amino group of the tether on the nucleoside sequence with a disuccinimidyl suberate tether. The disuccinimidyl suberate tether couples with the amine tether on the nucleoside to extend the size of the tether. The extended tether is then reacted with an amino group of the ligand molecule.

One aspect of the invention relates to a method of preparing a metal-conjugated oligonucleotide comprising the step of:

contacting a oligonucleotide comprising a metal-chelating ligand with a metal ion.

Another aspect of the invention relates to a method of preparing a metal-conjugated oligonucleotide comprising the step of:

contacting an oligonucleotide comprising a pendant nucleophile with a metal-chelating ligand comprising an electrophile, wherein said metal-chelating ligand comprising an electrophile is bound to a metal ion.

In certain embodiments, the present invention relates to the aforementioned methods, wherein said oligonucleotide is a siRNA, a microRNA, an antagomir, an antisense oligonucleotide, an aptamer, or a decoy nucleic acid.

In certain embodiments, the present invention relates to the aforementioned methods, wherein said oligonucleotide is a siRNA.

In certain embodiments, the present invention relates to any one of the aforementioned methods, wherein said metal-chelating ligand comprising an electrophile is a selected from the compounds of FIGS. 10 and 11.

In certain embodiments, the present invention relates to any one of the aforementioned methods, wherein said metal-chelating ligand comprising an electrophile is bound to M²; and M² is a paramagnetic metal ion or a lanthanide metal ion.

### Use in Signal Generation

The compounds and compositions of this disclosure can be used for signal generation. For example, europium(III) nanoparticle labels may be used for signal generation. The detection limits of the microtitration well and microparticle applications were 4.0×10⁵ and 6.1×10⁴ copies of target sequence (see Exemplification). The reference assay, based on the detection of Eu(III) chelate-labeled detection probes, had a detection limit of 8.5×10⁷ copies. The high sensitivity of the designed assays is based on the long-lifetime fluorescence of nanoparticle labels, on time-resolved fluorometry, and on signal rather than target amplification.

An example of a protocol for Eu(III) chelation is as follows: Step 1: Take amino-linked siRNA (500 nmol) in 100 mL of 0.2 M carbonate buffer (pH: 8.5 to 8.9); Step 2: Mix five fold molar excess of chelator NHS ester in dry DMF to amino-linked siRNA solution and leave at ambient temperature overnight; Step 3: Add molar equivalent of EuCl3 to the reaction mixture; Turbidity appears upon addition and was subsequently disappears during course of chelation; Leave at room temperature for 30 min with occasional mixing; Step 4: Dialyze the conjugated siRNAs using a dialysis membrane (3500 MWCO) from Spectrapor against water; and Step 5: Analyze the conjugated siRNA by Electrospray Ionization.

### Use in Magnetic Resonance Imaging

Magnetic Resonance Imaging (MRI), as a powerful diagnostic technique, has been widely used in medical and biological researches. Raymond, K. N., Pierre, V. C., Bioonj. Chem. 2005, 16, 3-8. In vivo, the differences in water distribution and relaxation time of the water protons can be effectively detected, thereby to produce high resolution, 3-D images of the tissue of interest. However, in practice to obtain high contrast medical images, often a Contrast Agent (CA) is necessary to enhance the specificity and sensitivity of MRI. And most of these CAs are complexes based on gadolinium (III) ion, which is highly paramagnetic with seven unpaired electrons. Caravan, P., Ellison, J. J., McMurry, T. J., Lauffer, R. B., Chem. Rev. 1999, 99, 2293-2352. Free Gd (III) ion is very toxic in vivo, and it is required to be complexed with strong organic chelators like macrocyclic polyaminocarboxylates before used on patients. Such Gd (III) chelates may be used as non-isotopic labels for siRNA targeting studies.

It follows that certain compounds and compositions of the disclosure which comprise a magnetic resonance imaging contrast component may be used in a similar manner as other MRI agents as described in U.S. Pat. No. 5,155,215, hereby incorporated by reference; U.S. Pat. No. 5,087,440, hereby incorporated by reference; Magn. Reson. Med., 1986, 3, 808; Radiology, 1988, 166, 835; and Radiology, 1988, 166, 69#. Generally, sterile aqueous solutions of the contrast agents are administered to a patient intravenously in dosages ranging from about 0.01 to about 1.0 mmoles per kg body weight.

As described above for Eu (III) chelated siRNA, a similar 3′-amino modified siRNA was synthesized. For the chelators, in certain embodiments, p-SCN-Bn-DOTA (S-2-(4-isothiocyanatobenzyl)-1,4,7,10-tetraazacyclododecane-tetraacetic acid) or DOTA-NHS-ester (1,4,7,10-tetraazacyclododecane-1,4,7,10-tetraacetic acid mono(N-hydroxysuccinimide ester) may be used; both of them can react with the 3′-amino modified siRNA to give DOTA conjugated siRNA at room temperature in mild basic conditions. When Gd(III) was added following the same conditions which were used for the Eu(III), a large amount of precipitate was observed while no desired product was detected using LC-MS. This is probably due to the stronger interactions of Gd(III) ions with phosphates on the siRNA backbone. Kumar, K., J. Alloys Compd., 1997, 249, 163-172.

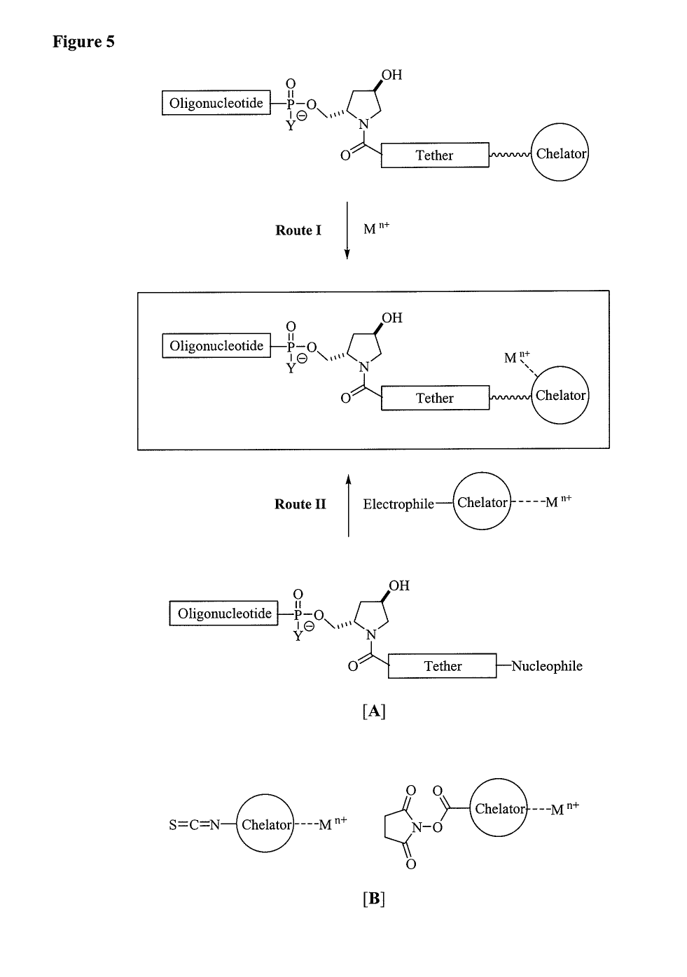

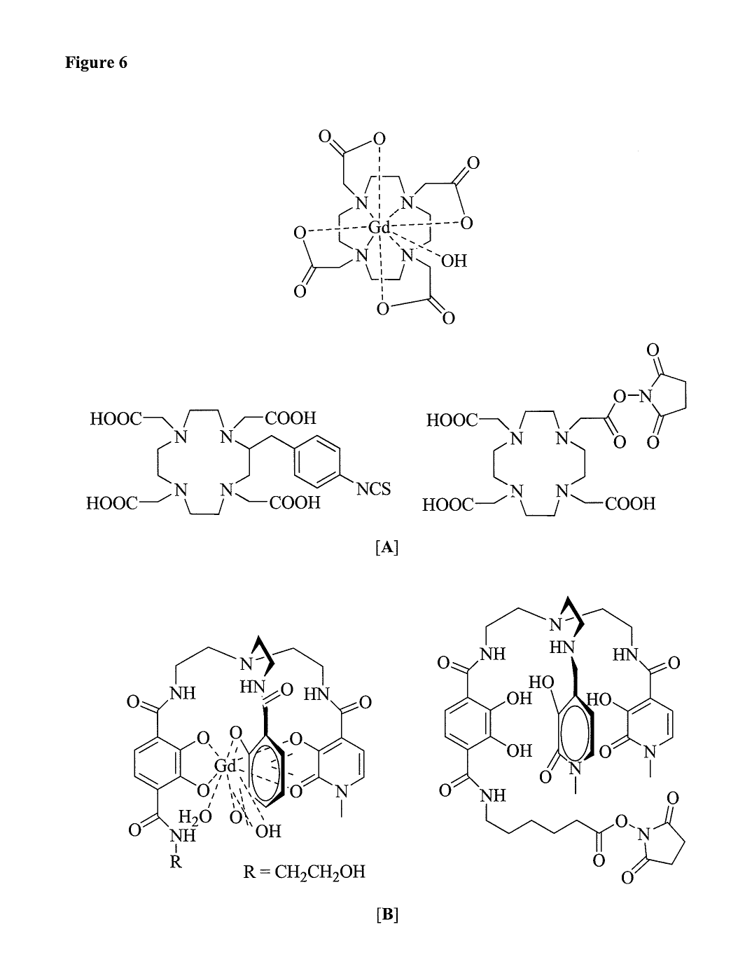

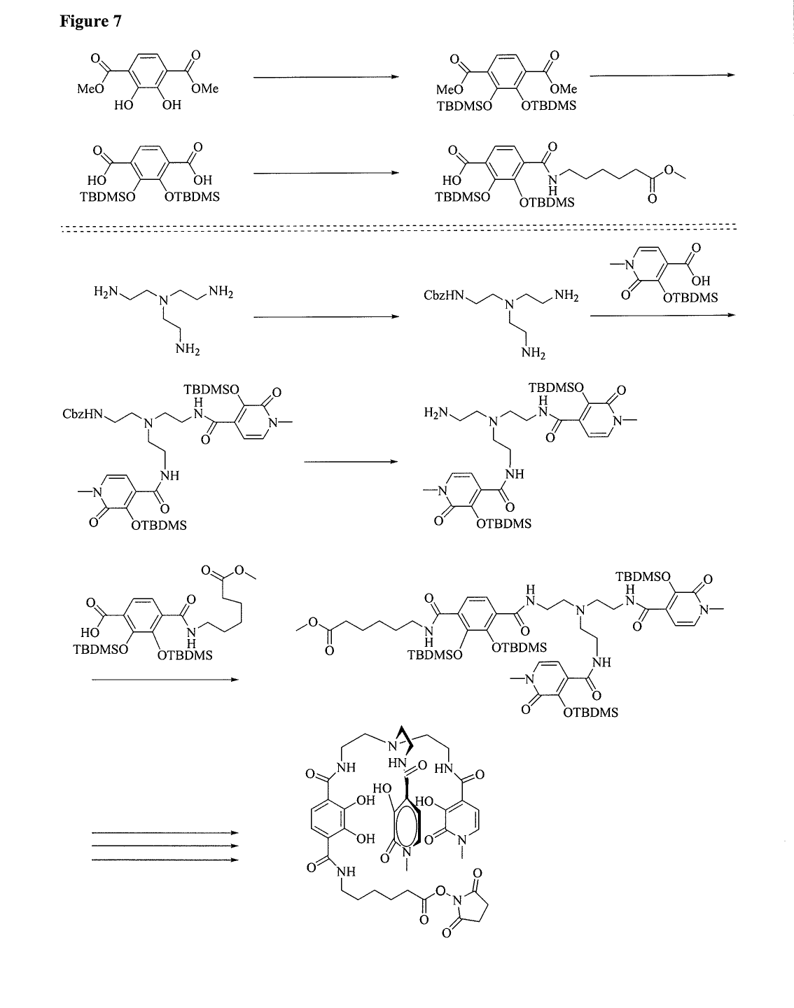

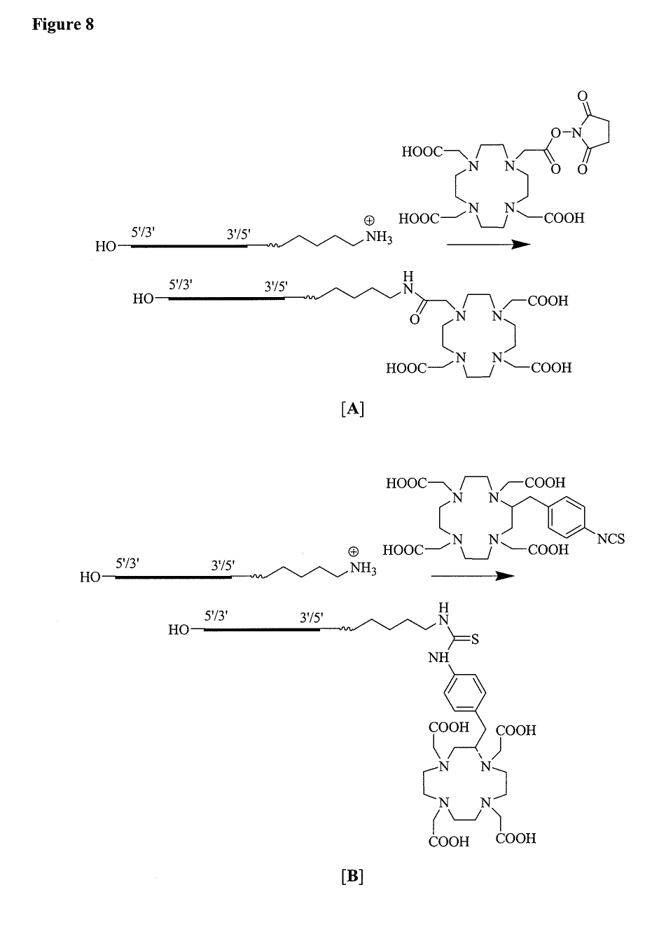

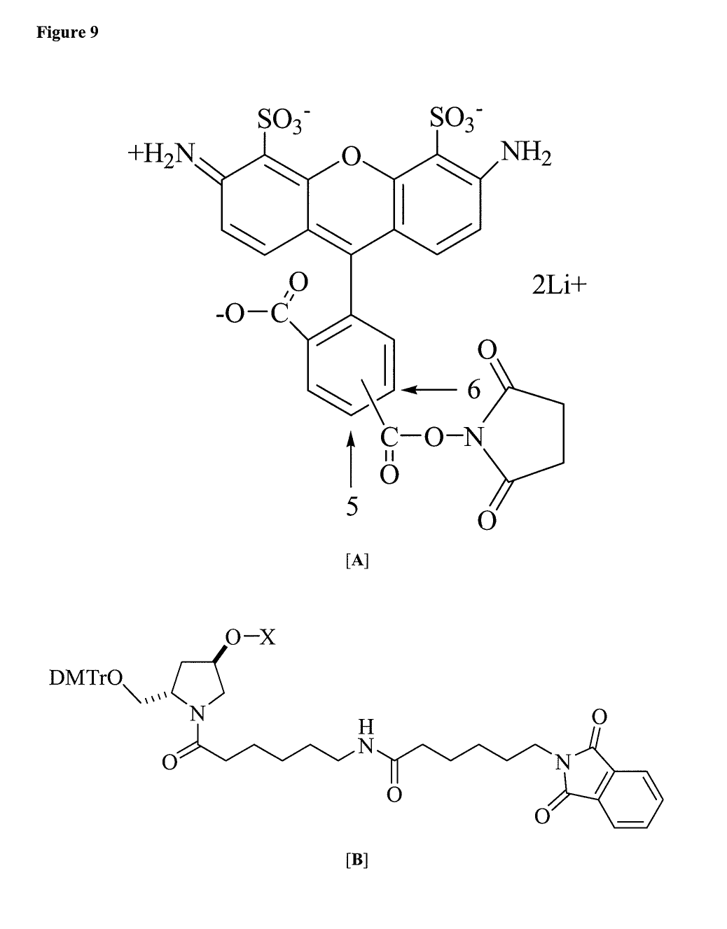

In order to avoid such precipitation problem, one can reverse the order of steps and prepare the Gd (III) complexed DOTA first, then to react with the 3′-amino modified siRNA. See FIG. 5. Hurskainen, P., Dahlen, P., Ylikoski, J., Kwiatkowski, M., Siitari, H., Lovgren, T., Nucl. Acids Res. 1991, 19, 1057-1061. For this new method, one starts with p-SCN-Bn-DOTA instead of DOTA-NHS-ester because p-SCN-Bn-DOTA is much more stable in water. Li, M., Selvin, P. R., Bioonj. Chem. 1997, 8, 127-132. Following this method, the Gd (III) complexed DOTA was prepared in an aqueous buffer, and then reacted with a 3′-amino modified siRNA.

In addition, the reaction conditions have been optimized by fine tuning their pHs using 0.2 M sodium carbonate buffer. Moreover, in order to minimize the presence of free Gd (III) ions, only 0.95 equiv of Gd³⁺ was used in the complexation with respect to the DOTA chelator. Finally, the conjugation reaction with siRNA was followed by SEC desalting twice using Sephadex G-25 (before and after reversed phase HPLC purification). Wen, X., Jackson, E. F., Price, R. E., Kim, E. E., Wu Q., Wallace, S., Charnsangavej, C., Gelovani, J. G., Li C., Bioonj. Chem. 2004, 15, 1408-1415.

### Use as a Luminescent Probe

Oligonucleotides of the invention can be conjugated to luminescent lathanide chelates, such as diethylenetriaminepentaacetic acid (DTPA) and DTPA-like chelates.

Luminescent lanthanide chelates have highly unusual spectral characteristics that make them useful nonisotopic alternatives to radioactive probes, as alternatives to organic fluorophores, particularly where there are problems of background autofluorescence, and as donors in luminescence resonance energy transfer. An isothiocyanate form of a lanthanide chelate which is highly luminescent when bound to terbium or europium has been synthesized. See Bioconjugate Chem. 1997, 8, 127-132. Linking of a chelate diethylenetriaminepentaacetic acid (DTPA), to a chromophore, 7-amino-4 methyl-2(1H)-quinolinone (cs124), and to L-p-aminophenylalanine, in which the aromatic amine was further converted to an isothiocyanate group was disclosed. It was also shown that site-specific attachments to triglycine and to the 5′ ends of amine-modified DNA oligomers could be made. In addition, as an alternative method of coupling to macromolecules, DTPA anhydride-cs124 was disclosed with a 5′ amine group on base-deprotected synthetic DNA oligomers. Synthesis and purification was relatively straightforward in both cases, and luminescent properties were favorable for several applications, including as nonisotopic labels, as long-lifetime alternatives to fluorophores in imaging and diagnostics and particularly as donors in luminescence resonance energy transfer. Compounds of the invention may similarly comprise a luminescent lanthanide chelate or chelates and be used in a similar manner.

### Methods of Use

One aspect of the invention relates to a method of following the bio-distribution of an oligonucleotide of the invention in an subject comprising the steps of: determining the site of concentration of a oligonucleotide in the subject by acquiring an image or images by a diagnostic imaging technique.

Another aspect of the invention relates to a method of detecting, imaging and/or monitoring disease in a patient comprising the steps of: acquiring an image of a site of concentration of the metal-conjugated oligonucleotide in the patient by a diagnostic imaging technique.

Another aspect of the invention relates to a method of cleaving a phosphate backbone linkage of a target oligonucleotide, comprising the steps of: contacting said target oligonucleotide with a oligonucleotide of the invention which is complementary to the target oligonucleotide.

### Kits

In certain aspects of the present disclosure is provided a kit comprising an oligonucleotide of the invention or a pharmaceutically acceptable salt thereof; a pharmaceutically acceptable carrier; and instructions for use thereof.

The terms “diagnostic kit” and “kit”, as used herein, refer to a collection of components in one or more vials that are used by the practicing end user in a clinical or pharmacy setting to synthesize diagnostic agents. The kit provides all the requisite components to synthesize and use the diagnostic agents (except those that are commonly available to the practicing end user such as water or saline for injection), such as a solution of the imaging agent or a precursor thereof, equipment for heating during the synthesis of the diagnostic agent, equipment necessary for administering the diagnostic agent to the patient such as syringes and shielding (if required), and imaging equipment.

Another aspect of the present disclosure is diagnostic kits for the preparation of diagnostic agents for detecting, imaging, and/or monitoring a pathological disorder. Diagnostic kits of the present disclosure comprise one or more vials containing the sterile, non-pyrogenic, formulation comprising a predetermined amount of a reagent of the present disclosure, and optionally other reducing agents, transfer ligands, buffers, lyophilization aids, stabilization aids, solubilization aids and bacteriostats

The inclusion of one or more optional components in the formulation will frequently improve the ease of synthesis of the diagnostic agent by the practicing end user, the ease of manufacturing the kit, the shelf-life of the kit, or the stability and shelf-life of the imaging agent. The one or more vials that contain all or part of the formulation can independently be in the form of a sterile solution or a lyophilized solid.

The inclusion of one or more optional components in the formulation will frequently improve the ease of synthesis of the diagnostic agent by practicing end user, the ease of manufacturing the kit, the shelf-life of the kit, or the stability and shelf-life of the imaging agent. The improvement achieved by the inclusion of an optional component in the formulation must be weighed against the added complexity of the formulation and added cost to manufacture the kit. The one or more vials that contain all or part of the formulation can independently be in the form of a sterile solution or a lyophilized solid.

Buffers useful in the preparation of diagnostic agents and kits thereof include but are not limited to phosphate, citrate, sulfosalicylate, and acetate. A more complete list can be found in the United States Pharmacopeia.

Lyophilization aids useful in the preparation of diagnostic agents and kits thereof include but are not limited to mannitol, lactose, sorbitol, dextran, Ficoll, and polyvinylpyrrolidine (PVP).

Stabilization aids useful in the preparation of of diagnostic agents and kits thereof include but are not limited to ascorbic acid, cysteine, monothioglycerol, sodium bisulfite, sodium metabisulfite, gentisic acid, and inositol.

Solubilization aids useful in the preparation of diagnostic agents and kits thereof include but are not limited to ethanol, glycerin, polyethylene glycol, propylene glycol, polyoxyethylene sorbitan monooleate, sorbitan monoloeate, polysorbates, poly(oxyethylene)-poly(oxypropylene)poly(oxyethylene) block copolymers (Pluronics) and lecithin. Typical solubilizing aids are polyethylene glycol, and Pluronics copolymers.

Bacteriostats useful in the preparation of of diagnostic agents and kits thereof include but are not limited to benzyl alcohol, benzalkonium chloride, chlorbutanol, and methyl, propyl or butyl paraben

A component in a diagnostic kit can also serve more than one function. A reducing agent can also serve as a stabilization aid, a buffer can also serve as a transfer ligand, a lyophilization aid can also serve as a transfer, ancillary or coligand and so forth.

The predetermined amounts of each component in the formulation are determined by a variety of considerations that are in some cases specific for that component and in other cases dependent on the amount of another component or the presence and amount of an optional component. In general, the minimal amount of each component is used that will give the desired effect of the formulation. The desired effect of the formulation is that the practicing end user can synthesize the diagnostic agent and have a high degree of certainty that the diagnostic agent can be injected safely into a patient and will provide diagnostic information about the disease state of that patient.

The diagnostic kits of the present disclosure can also contain written instructions for the practicing end user to follow to synthesize the diagnostic agents. These instructions may be affixed to one or more of the vials or to the container in which the vial or vials are packaged for shipping or may be a separate insert, termed the package insert.

Metallopharmaceuticals for use as magnetic resonance imaging contrast agents are provided to the end user in their final form in a formulation contained typically in one vial, as either a lyophilized solid or an aqueous solution. The end user reconstitutes the lyophilized solid with water or saline and withdraws the patient dose or simply withdraws the dose from the aqueous solution formulation as provided.

### Definitions

For convenience, certain terms employed in the specification, examples, and appended claims are collected here.

As used herein, the singular forms “a”, “an”, and “the” include plural reference unless the context clearly dictates otherwise.

As used herein, the term “bacteriostat” means a component that inhibits the growth of bacteria in a formulation either during its storage before use of after a diagnostic kit is used to synthesize a diagnostic agent.

The term “buffer,” as used herein, refers to a substance used to maintain the pH of the reaction mixture from about 3 to about 10.

The term “carrier”, as used herein, refers to an adjuvant or vehicle that may be administered to a patient, together with the compounds and/or diagnostic agents of this disclosure which does not destroy the activity thereof and is non-toxic when administered in doses sufficient to deliver an effective amount of the diagnostic agent and/or compound.

As used herein, the phrase “donor atom” refers to the atom directly attached to a metal by a chemical bond.

As used herein, the term “lyophilization aid” means a component that has favorable physical properties for lyophilization, such as the glass transition temperature, and is added to the formulation to improve the physical properties of the combination of all the components of the formulation for lyophilization.

As used herein, the term “metallopharmaceutical” means a pharmaceutical comprising a metal. The metal is the origin of the imageable signal in diagnostic applications.

As used herein, the term “reagent” means a compound of this disclosure capable of direct transformation into a diagnostic agent of this disclosure. Reagents may be utilized directly for the preparation of the diagnostic agents of this disclosure or may be a component in a kit of this disclosure.

As used herein, the phrase “solubilization aid” is a component that improves the solubility of one or more other components in the medium required for the formulation.

The term “stable”, as used herein, refers to compounds and/or diagnostic agents which possess the ability to allow manufacture and which maintain their integrity for a sufficient period of time to be useful for the purposes detailed herein. Typically, the compounds and/or diagnostic agents of the present disclosure are stable at a temperature of 40° C. or less in the absence of moisture or other chemically reactive conditions for at least a week.

As used herein, the phrase “stabilization aid” means a component that is added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting with species that degrade other components or the metallopharmaceutical.

The term “sterile,” as used herein, means free of or using methods to keep free of pathological microorganisms.

The term “silence” means to at least partially suppress. For example, in certain instances, the gene is suppressed by at least about 25%, 35%, or 50% by administration of the double stranded oligonucleotide of the invention. In a embodiment, the gene is suppressed by at least about 60%, 70%, or 80% by administration of the double stranded oligonucleotide of the invention. In a more embodiment, the gene is suppressed by at least about 85%, 90%, or 95% by administration of the double stranded oligonucleotide of the invention. In a most embodiment, the gene is suppressed by at least about 98% or 99% by administration of the double stranded oligonucleotide of the invention.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl(alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl, cyano, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, fluoroalkyl, cyano, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, fluoroalkyl, cyano, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on page 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

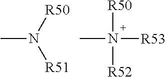

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

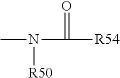

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

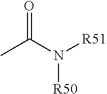

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

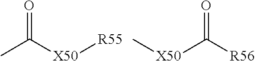

wherein X50 is a bond or represents an oxygen or a sulfur, and R55 and R56 represents a hydrogen, an alkyl, an alkenyl, —(CH₂)ₘ—R61or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

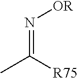

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

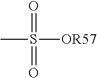

in which R57 is an electron pair, hydrogen, alkyl, cycloalkyl, or aryl.

The term “sulfate” is art recognized and includes a moiety that may be represented by the general formula:

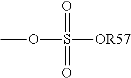

in which R57 is as defined above.

The term “sulfonamido” is art recognized and includes a moiety that may be represented by the general formula:

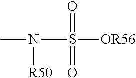

in which R50 and R56 are as defined above.

The term “sulfamoyl” is art-recognized and refers to a moiety that may be represented by the general formula:

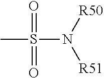

in which R50 and R51 are as defined above.

The term “sulfonyl” is art-recognized and refers to a moiety that may be represented by the general formula:

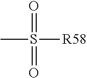

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

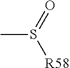

in which R58 is defined above.

The term “phosphoryl” is art-recognized and may in general be represented by the formula:

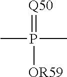

wherein Q50 represents S or O, and R59 represents hydrogen, a lower alkyl or an aryl. When used to substitute, e.g., an alkyl, the phosphoryl group of the phosphorylalkyl may be represented by the general formulas:

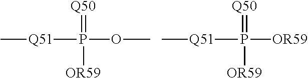

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

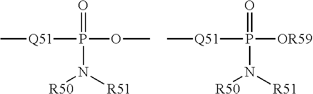

wherein Q51, R50, R51 and R59 are as defined above.

The term “phosphonamidite” is art-recognized and may be represented in the general formulas:

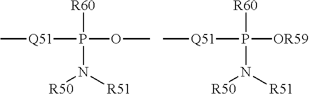

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The term “alkenylene,” as used herein, refers to a divalent group of derived from a straight or branched chain hydrocarbon containing from two to fourteen carbon atoms at least one carbon-carbon double bond.

The term “alkylene,” as used herein, refers to a divalent group derived from a straight or branched chain saturated hydrocarbon of one to fourteen carbon atoms.

The term “arylalkylene,” as used herein, refers to a divalent arylalkyl group, where one point of attachment to the parent molecular moiety is on the aryl portion and the other is on the alkyl portion.

The term “arylene,” as used herein, refers to a divalent aryl group.

The term “cycloalkylene,” as used herein, refers to a divalent cycloalkyl group.

The term “heteroalkylene,” as used herein, refers to an alkylene group wherein one to seven of the carbon atoms are replaced by a heteroatom selected from O, NH, and S.

The term “heterocyclylalkylene,” as used herein, refers to a divalent heterocyclylalkyl group, where one point of attachment to the parent molecular moiety is on the heterocyclyl portion and the other is on the alkyl portion.

The term “heterocyclylene,” as used herein, refers to a divalent heterocyclyl group.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

In some instances, the number of carbon atoms in any particular group is denoted before the recitation of the group. For example, the term “C₆₋₁₀aryl” denotes an aryl group containing from six to ten carbon atoms, and the term “C₆₋₁₀aryl-C₁₋₁₀alkyl,” refers to an aryl group of six to ten carbon atoms attached to the parent molecular moiety through an alkyl group of one to ten carbon atoms. Where these designations exist they supercede all other definitions contained herein.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

When any variable occurs more than one time in any substituent or in any formula, its definition on each occurrence is independent of its definition at every other occurrence. Thus, for example, if a group is shown to be substituted with 0-2 R²³, then said group may optionally be substituted with up to two R²³, and R²³ at each occurrence is selected independently from the defined list of possible R^{2#.} Also, by way of example, for the group —N(R²⁴)₂, each of the two R²⁴ substituents on the nitrogen is independently selected from the defined list of possible R²⁴. Combinations of substituents and/or variables are permissible only if such combinations result in stable compounds and/or diagnostic agents. When a bond to a substituent is shown to cross the bond connecting two atoms in a ring, then such substituent may be bonded to any atom on the ring

It should also be understood that the compounds and/or diagnostic agents of this disclosure may adopt a variety of conformational and ionic forms in solution, in pharmaceutical compositions and in vivo. Although the depictions herein of specific compounds and/or diagnostic agents of this disclosure are of particular conformations and ionic forms, other conformations and ionic forms of those compounds and/or diagnostic agents are envisioned and embraced by those depictions.

Further, this disclosure is intended to encompass compounds having formula (I) when prepared by synthetic processes or by metabolic processes including those occurring in the human or animal body (in vivo) or processes occurring in vitro.

### EXEMPLIFICATION

Other features of the disclosure will become apparent in the course of the following descriptions of exemplary embodiments which are given for illustration of the disclosure and are not intended to be limiting thereof. The present disclosure will now be illustrated by reference to the following specific, non-limiting examples. Those skilled in the art of organic synthesis may be aware of still other synthetic routes to the disclosure compounds and/or diagnostic agents. The reagents and intermediates used herein are either commercially available or prepared according to standard literature procedures, unless otherwise described.

### Example 1

### Amino-Modified Oligonucleotides for Metal Chelation

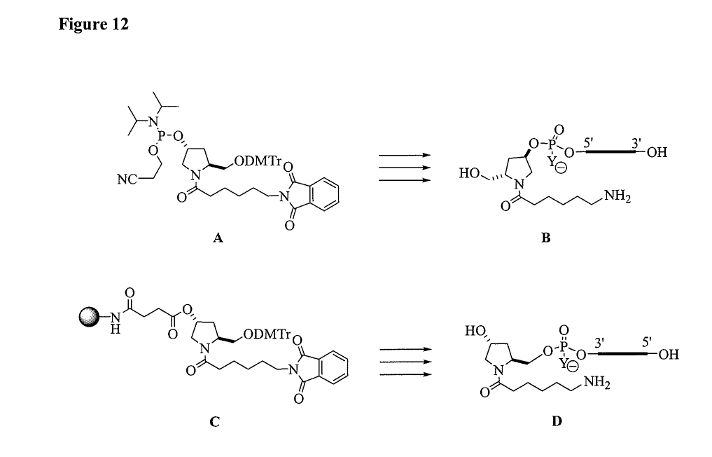

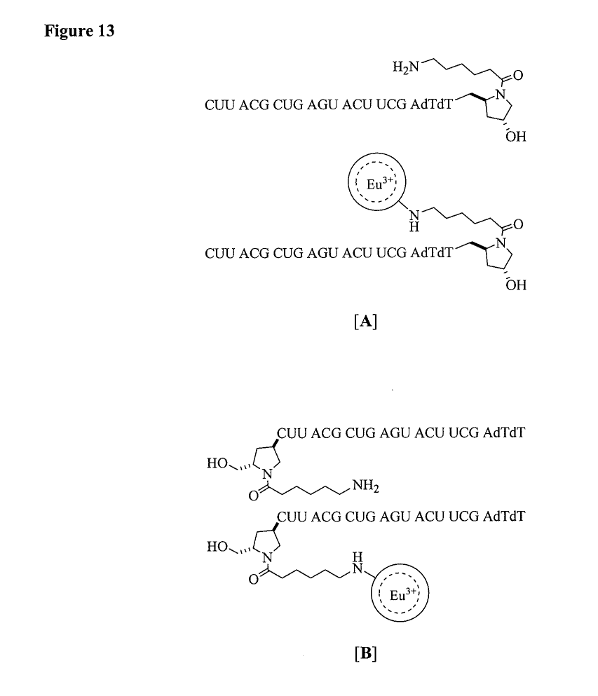

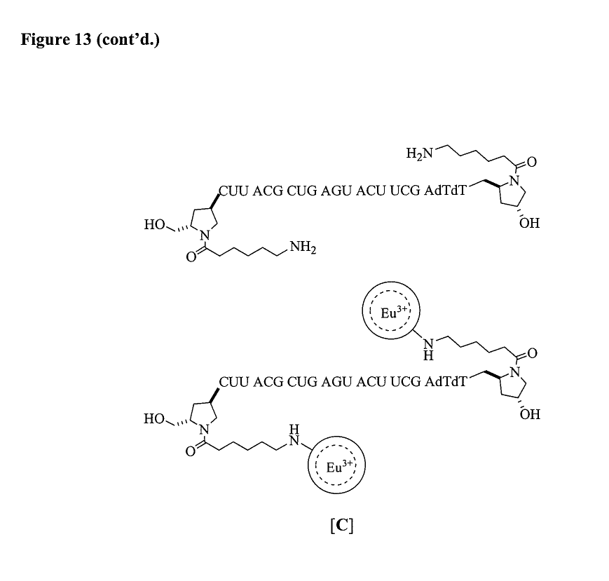

The 5′-and 3′-amino modified oligonucleotides (RNA, DNA, antisense oligonucleotide, etc.) were individually synthesized using commercially available 5′-O-(4,4′-dimethoxytrityl)-2′-O-t-butyldimethylsilyl-3′-O-(2-cyanoethyl-N,N-diisopropyl) RNA phosphoramidite monomers of 6-N-benzoyladenosine (A^{Bz}), 4-N-acetylcytidine (C^{Ac}), 2-N-isobutyrylguanosine (G^{iBu}), and uridine (U), according to standard solid phase oligonucleotide synthesis protocols as previously described. Krutzfeldt, J., Rajewsky, N., Braich, R., Rajeev, K. G., Tuschl, T., Manoharan, M. and Stoffel, M. “Silencing of microRNAs in vivo with ‘antagomirs’,” Nature 2005, 438, 685. 0.2 M Phenyl acetyl disulfide (PADS) in 1:1 3-picoline:acetonitrile was used as an oxidant to obtain the phosphorothioate backbone modification. The 5′- and 3′-amino modified oligonucleotides B and D were synthesized from corresponding hydroxyprolinol-phthalimido phosphoramidite A and solid support C, respectively, as shown in FIG. 12. Iyer, R. P., Eagen, W., Regan, J. B. Beaucage, S. L. “3H-1,2-benzodithiole-3-one-1,2-dioxide as an improved sulfurizing agent in the solid-phase synthesis of oligodeoxyribonucleoside phosphorothioates,” J. Am. Chem. Soc. 1990, 112, 1253-1354; and Manoharan, M., Kesavan, V., and Rajeev, K. G. “SiRNA's containing ribose substitutes to which lipophilic moieties may be attached,” U.S. Pat. Appl. Publ. 2005/107325; hereby incorporated by references in its entirety. The sequences were purified by high-performance liquid chromatography (HPLC) on an in-house packed TSK gel ion exchange column. The buffers were 20 mM sodium phosphate in 10% CH₃CN, pH 8.5 (buffer A) and 20 mM Sodium phosphate in 10% CH₃CN, 1M NaBr, pH 8.5 (buffer B). Fractions containing full-length oligonucleotides were pooled, desalted and lyophilized. Analytical HPLC, CGE and ES LC-MS established the integrity of the compounds.

### Example 2A

### Europium (III) Labeling of Amine-Activated RNA

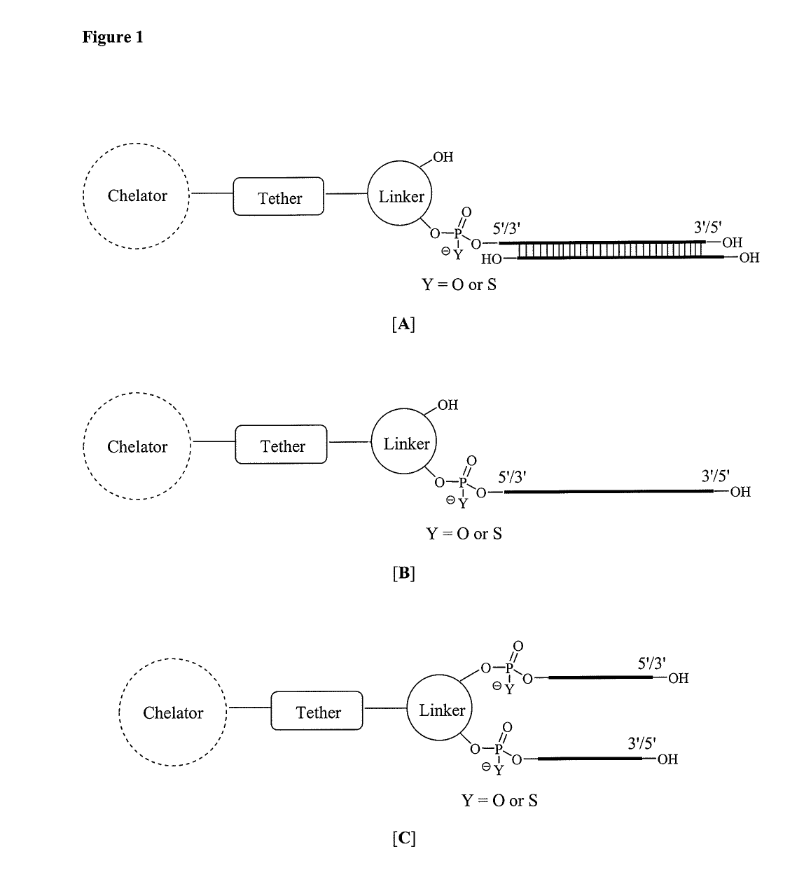

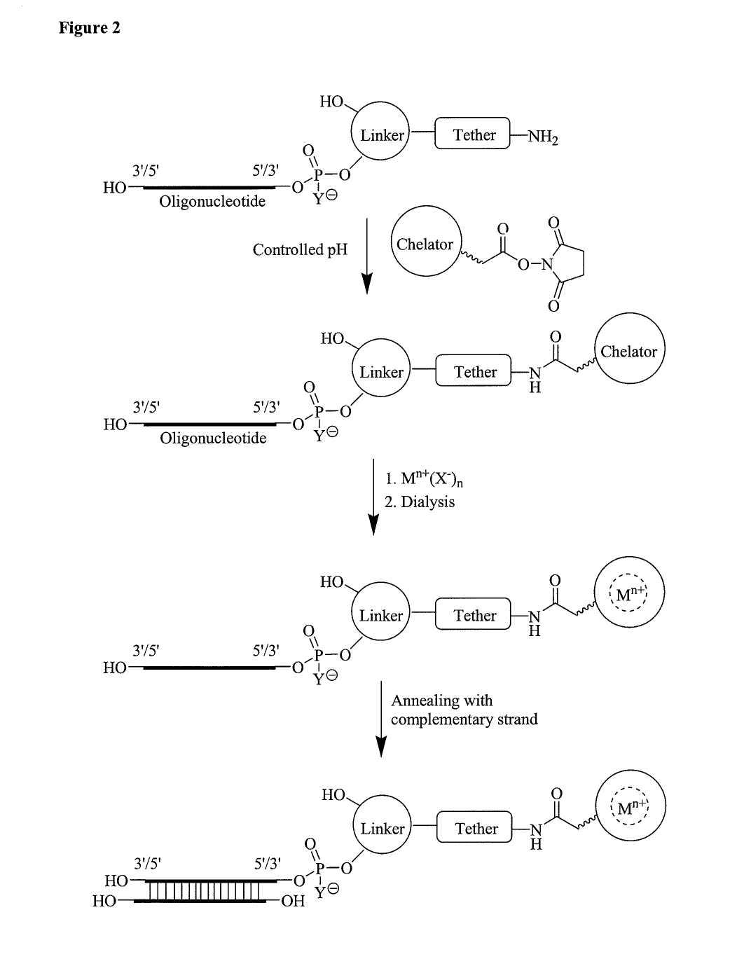

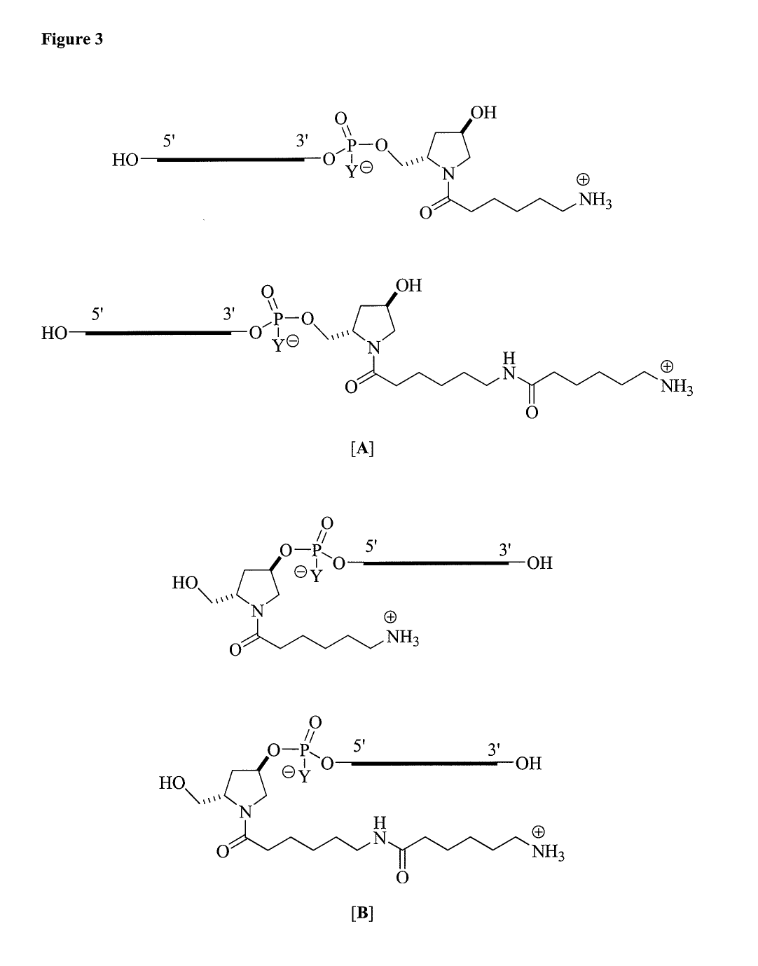

To incorporate the europium (Eu³⁺) group at the 5′-end and 3′-end of the oligonucleotide, 500 nmol of amino-modified oligonucleotides were dissolved in 100 μl of 0.2 M sodium carbonate buffer pH 8.9. The proprietary chelate (proprietary chelate was received from BIOPAL, Worcester, Mass.) was reconstituted in 50 μl of dry DMF. A 12 fold molar excess of chelate was added to the amine modified siRNA. The pH was adjusted to 9.1 for 15 min. The mixture was kept at room temperature overnight, with occasional mixing. An equivalent molar amount of europium (1 M EuCl₃, 0.2 ml, 152 mg Eu/mL) was added to the chelated RNA. Initially a precipitate is formed, but it disappears as the europium is chelated. The mixture was left at room temperature for 30 minutes with occasional mixing. The labeled siRNAs was dialyzed using a dialysis membrane (3500 MWCO) from Spectrapor against water. Analysis of europium-labeled siRNA was performed on an Agilent HPLC using Dionex 4*250 mm PA-100 column. Buffer A was 20 mM sodium phosphate in 10% CH₃CN, pH 8.5, buffer B was 20 mM sodium phosphate in 10% CH₃CN, 1M NaBr, pH 8.5. Separation was performed on a 0-65% B segmented gradient with buffers and column heated to 65° C. Analytical anion exchange HPLC, CGE and ES-LC-MS confirmed the integrity of the compounds. See FIG. 1#.

### Example 2B

### Europium (III) Labeled siRNA

The Eu(III) labeled oligonucleotide from Example 2 was annealed with complementary guide strand or passenger strand to obtain the corresponding siRNA. The annealing was performed as reported in the prior arts. See Manoharan, M., Kesavan, V., and Rajeev, K. G. “SiRNA's containing ribose substitutes to which lipophilic moieties may be attached,” U.S. Pat. Appl. Publ. 2005/107325.

### Example 3A

### Gadolinium (III) Chelated p-SCN-Bn-DOTA

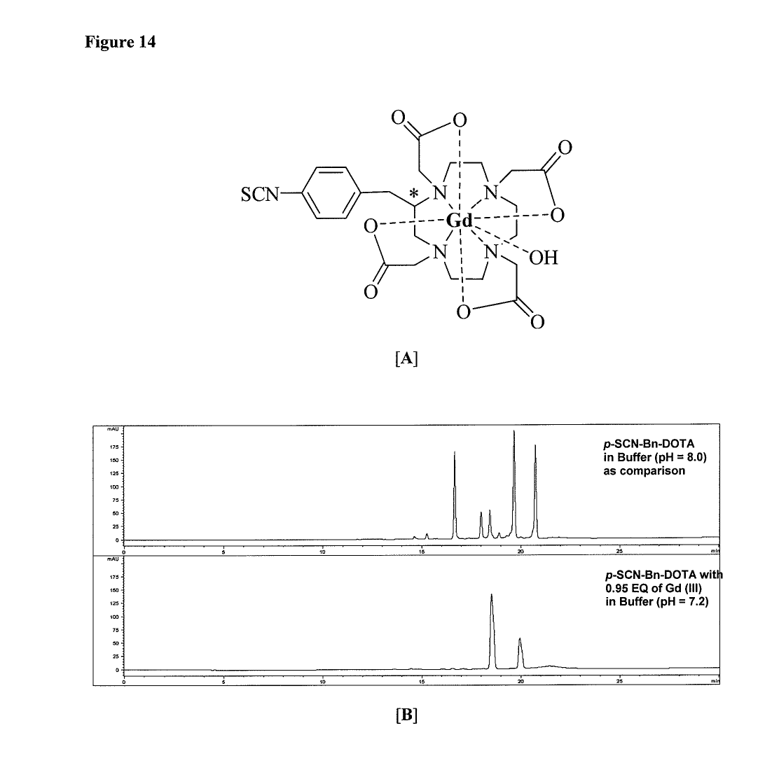

158 mg of p-SCN-Bn-DOTA (286 μmol, 20 equiv) was dissolved in 9.0 mL of a 0.2 M sodium carbonate buffer (pH=8.9). Mishra, A., Pfeuffer, J., Mishra, R., Engelmann, J., Mishra, A. K., Ugurbil, K., Logothetis, N. K., Bioonj. Chem. 2006, 17, 773-780. Some air bubbles formed during dissolvation and the solution's pH changed to 8.0. Mishra, A., Pfeuffer, J., Mishra, R., Engelmann, J., Mishra, A. K., Ugurbil, K., Logothetis, N. K., Bioonj. Chem. 2006, 17, 773-780; and Rudovsky, J., Kotek, J., Hermann, P., Lukes, I., Mainero, V., Aime, S., Org. Biomol. Chem., 1995, 3, 112-117. 272 μL of a 1.0 M GdCl₃ (272 μmol, 19 equiv) aqueous solution was added. First a large amount of white precipitate was observed, and after 15 min of vortexing, most of the precipitate disappeared and gave a solution with small amount of white solid present. Lu, Z., Wang, X., Parker, D. L., Goodrich, K. C., Buswell, H. R., Bioonj. Chem. 2003, 14, 715-719. This mixture was filtered through a 0.2 μm PTFE filter disc and followed by washings twice with 1.0 mL of sodium carbonate buffer (pH=8.9) each. The resulting solution was then checked by reversed phase HPLC to yield the compound shown in FIG. 14.

### Example 3B

### Gadolinium (III) Complexed siRNA Through p-SCN-Bn-DOTA

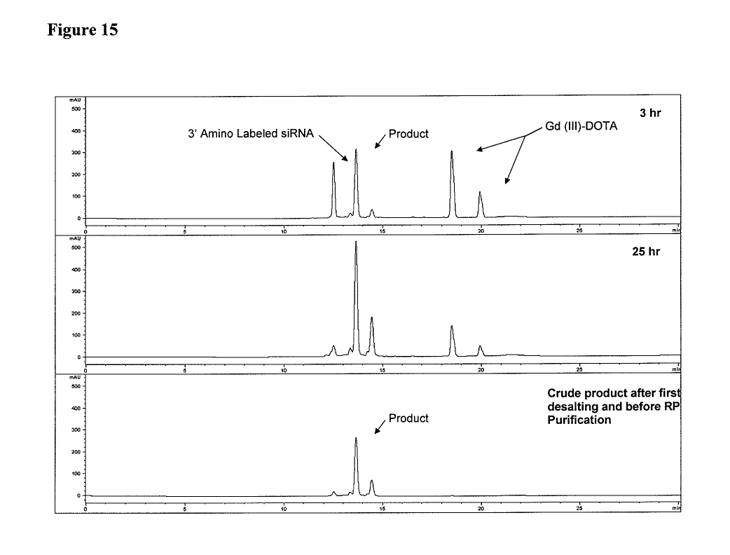

100 mg of 3′ amino modified siRNA (AL-SQ 3679) (14.3 μmol, 1.0 equiv) was dissolved in 1.5 mL of deionized water. Then the Gd (III) chelated p-SCN-Bn-DOTA solution was added to the siRNA. The pH of the resulting mixture was adjusted from 8.2 to 8.8 using 1.0 M NaOH solution. Then the reaction mixture was left at room temperature, and it was checked three times by reversed phase HPLC after 3 hr, 21 hr and 25 hr, respectively. See FIG. 15.

Once the reaction was complete, the mixture was desalted using a 5-cm Sephadex G-25 column on an Akta prime system to give the crude product. It was followed by purification on a Waters HPLC using a 2-cm Source 15 column. Collected fractions were concentrated and desalted again to give the final product (50.0 mg) in 45% of the yield based on siRNA.

### Example 3C

### Gadolinium (III) Labeled siRNA

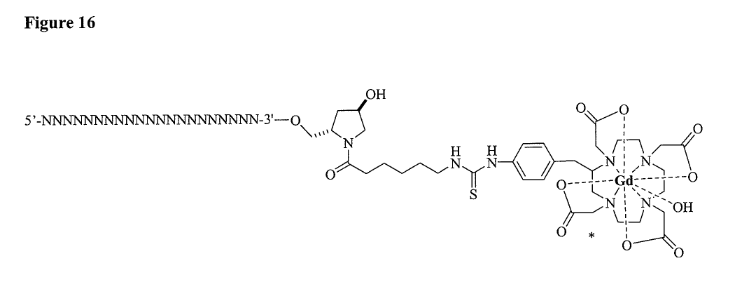

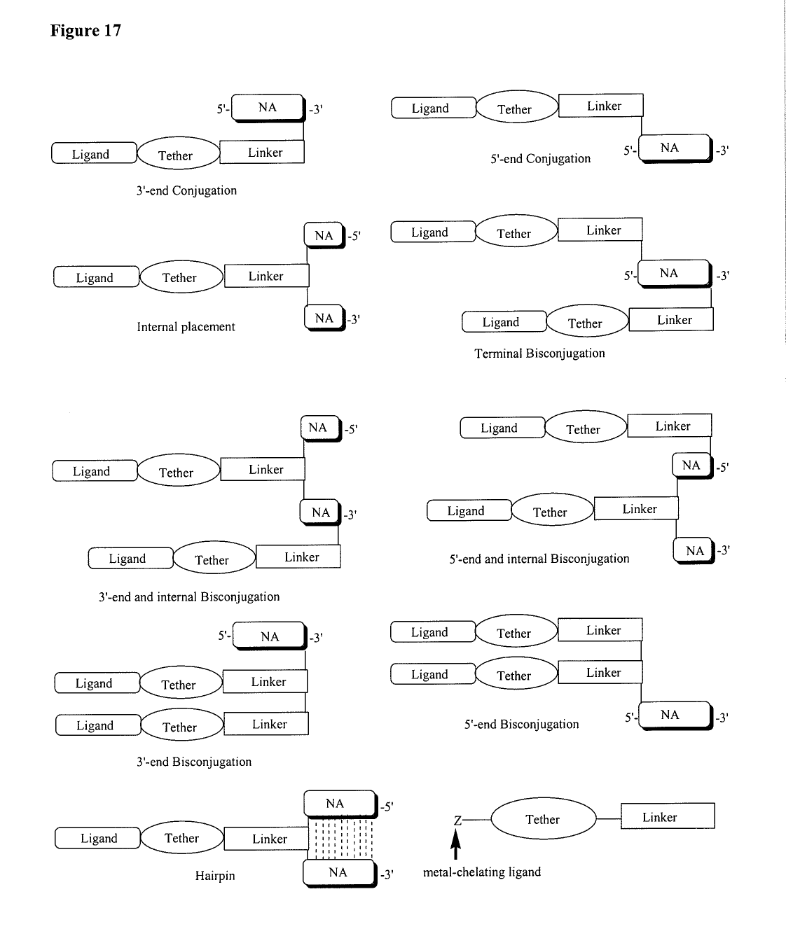

The Gd(III) labeled oligonucleotide from Example 3B was annealed with complementary guide strand or passenger strand to obtain the corresponding siRNA. The annealing was performed as reported in Manoharan, M., Kesavan, V., and Rajeev, K. G. “SiRNA's containing ribose substitutes to which lipophilic moieties may be attached,” U.S. Pat. Appl. Publ. 2005/107325. See FIG. 16.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-30. (canceled)

31. An oligonucleotide, or a pharmaceutically-acceptable salt thereof, comprising at least one strand represented by formula (I): 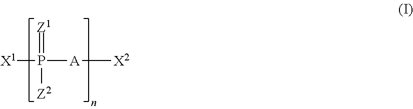
wherein, independently for each occurrence,
A is 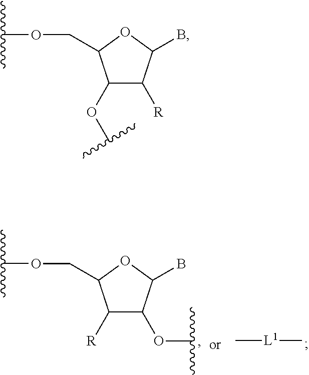
B is 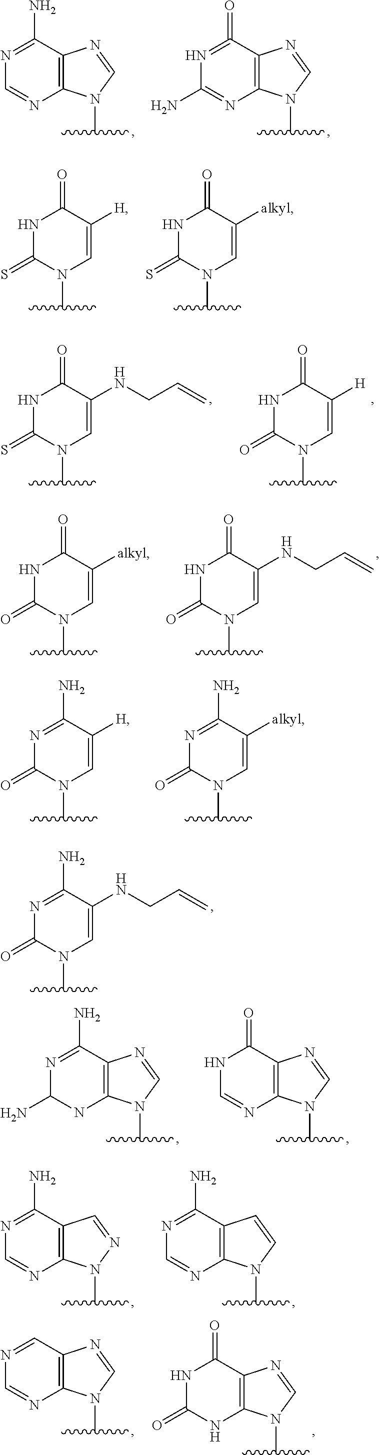 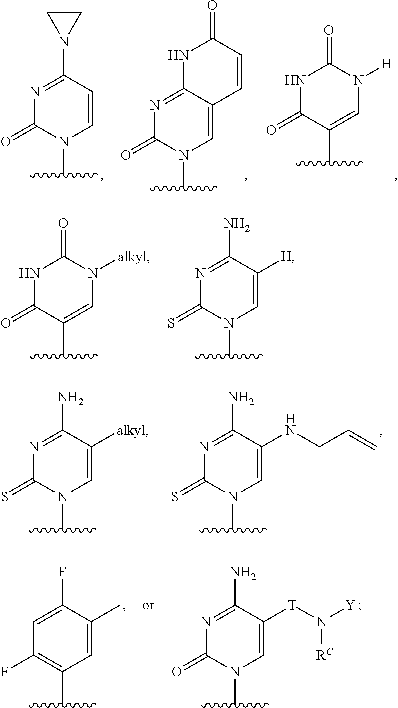
X¹ is —OR^{A}, 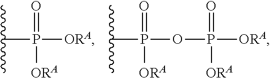
or -L²;
X² is —H, —R^{B}, 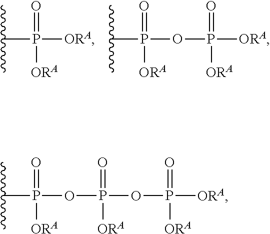
or -L²;
Z¹ is O or S;
Z² is —OH, —OM¹, —O-alkyl, —O-aryl, —O-aralkyl, —SH, —SM¹, —S-alkyl, —S-aryl, —S-aralkyl, —N(R^{E})₂, —(C(R^{E})₂)ₘ—N(R^{E})₂, —N(R^{E})—(C(R^{E})₂)ₘ—N(R^{E})₂, or alkyl;
L¹ is 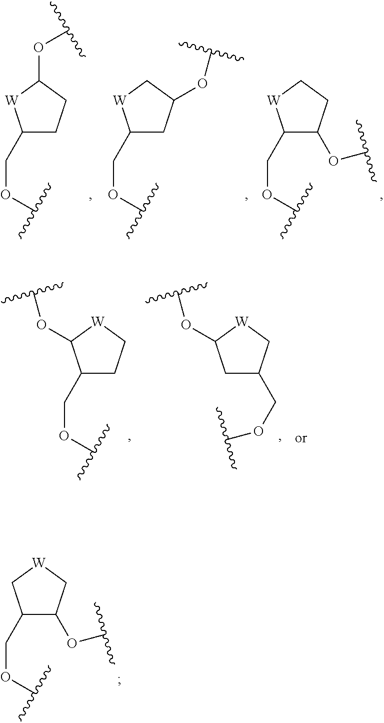
L² is 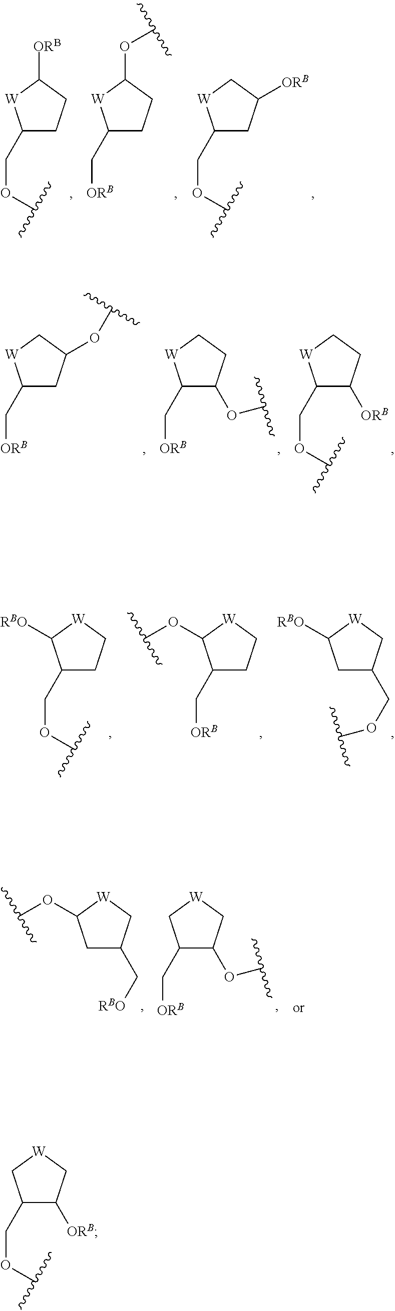
W is NC(═O)-T-N(R^{C})Y, or N-T-N(R^{C})Y;
T is —R^{D}—, —R^{D}—N(R^{C})—R^{D}— or —R^{D}—N(R^{C})C(═O)—R^{D}—;
Y is —H or —Z;
Z is a metal-chelating ligand or a metal-chelating ligand bound to M²;
R is —H, —OH, —F, —Cl, —Br, —O-alkyl, —O-allyl, —O—(C(R^{E})₂)ₘ—OR^{E}, —O—(C(R^{E})₂)ₘ—SR^{E}, —O—(C(R^{E})₂)ₘ—N(R^{E})₂, —O—(C(R^{E}₂)ₘ—C(O)N(R^{E})₂, —N(R^{E})₂, —S(C₁-C₆)alkyl, —O—(C(R^{E})₂)ₘ—O—(C₁-C₆)alkyl, —O—(C(R^{E})₂)ₘ—S(C₁-C₆)alkyl, —O(C(R^{E})₂)ₘ—O—(C(R^{E})₂)ₘ—N((C₁-C₆)alkyl)₂, or —O—(C(R^{E})₂)ₘ—O—N((C₁-C₆)alkyl)₂;
R^{A} is hydrogen, M¹ or alkyl;
R^{B} is hydrogen, alkyl, aryl, aralkyl, acyl or silyl;
R^{C} is hydrogen, alkyl, acyl, or aralkyl;
R^{D} is alkylene, heteroalkylene, alkenylene, alkynylene, arylene, or aralkylene;
R^{E} is hydrogen or alkyl; or two R^{E} taken together with the atoms to which they are bound form a 3-, 4-, 5-, 6-, or 7-membered carbocyclic or heterocyclic ring;
n is 0-200 inclusive;
m is 1-4 inclusive;
M¹ comprises an alkali metal ion or a transition metal ion with an overall charge of +1; and
M² is a paramagnetic metal ion or a lanthanide metal ion.

32. The oligonucleotide of claim 31, wherein the oligonucleotide is a siRNA, a microRNA or an antagomir.

33. The oligonucleotide of claim 31, wherein the oligonucleotide is a double-stranded oligonucleotide.

34. The oligonucleotide of claim 31, wherein n is 8-30 inclusive.

35. The oligonucleotide of claim 34, wherein n is 10-25 inclusive.

36. The oligonucleotide of claim 35, wherein n is 17-23 inclusive.

37. The oligonucleotide of claim 36, wherein n is 20.

38. The oligonucleotide of claim 35, wherein M² is Gd(III), Mn(II), Mn(III), Cr(II), Cr(III), Cu(II), Fe (III), Pr(III), Nd(III), Sm(III), Tb(III), Yb(III), Dy(III), Ho(III), Eu(II), Eu(III), or Er(III).

39. The oligonucleotide of claim 38, wherein M² is Gd(III), Cu(II), or Eu(III).

40. The oligonucleotide of claim 31, wherein Z is selected from a compound represented by formula (II) or (III): 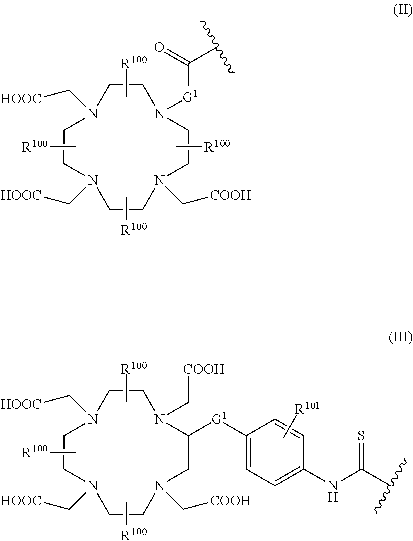
wherein, independently for each occurrence,
G¹ is alkylene, heteroalkylene, alkenylene, alkynylene, arylene, or aralkylene;
R¹⁰⁰ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

41. The oligonucleotide of claim 31, wherein Z is selected from the group consisting of: 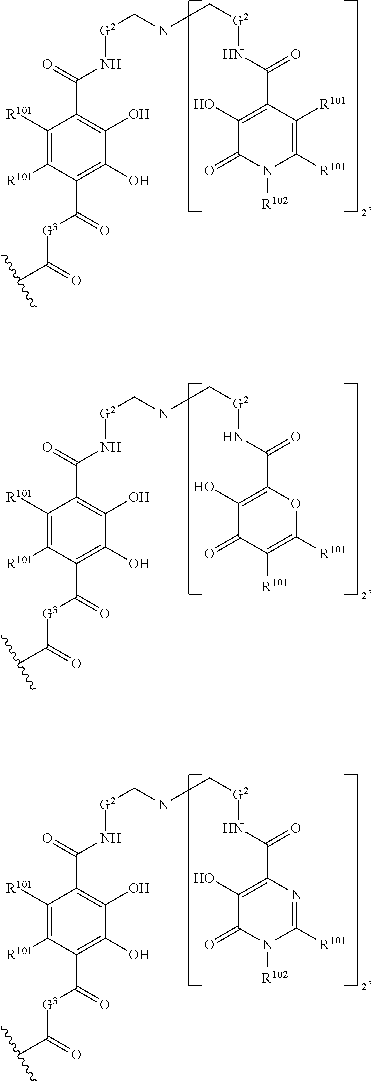 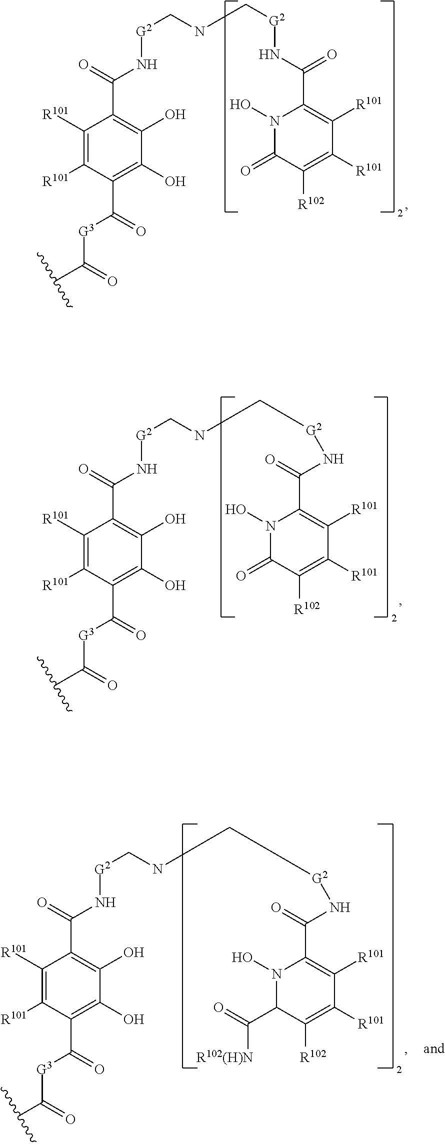 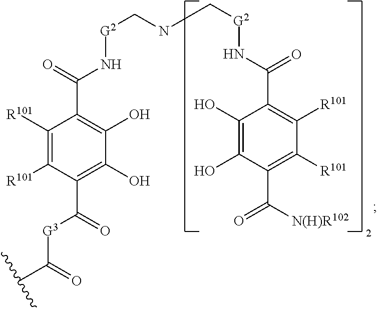
wherein:
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰² is hydrogen, alkyl, alkenyl, alkynyl, cycloalkyl, heterocyclyl, aryl, aralkyl, heteroaryl, heteroaralkyl, or fluoroalkyl;
G² is alkylene; and
G³ is alkylene or heteroalkylene.

42. The oligonucleotide of claim 31, wherein Z is selected from the group consisting of: 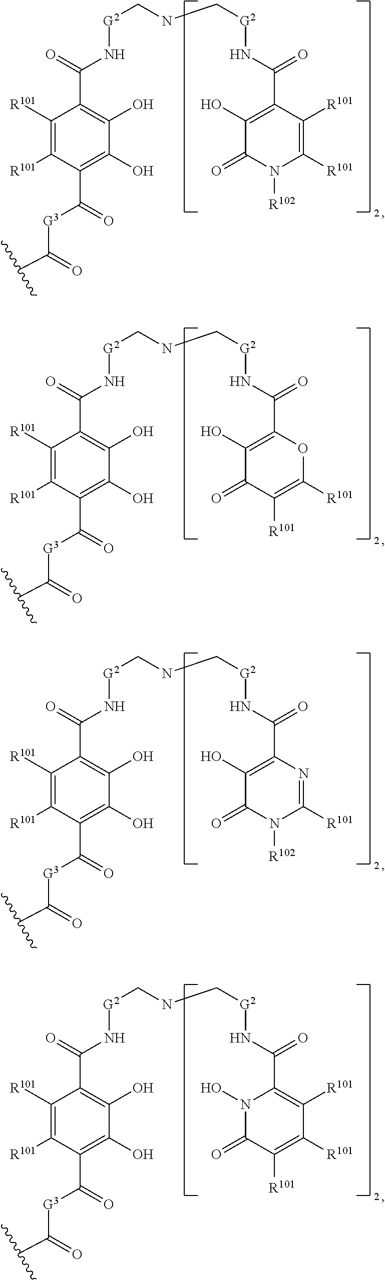 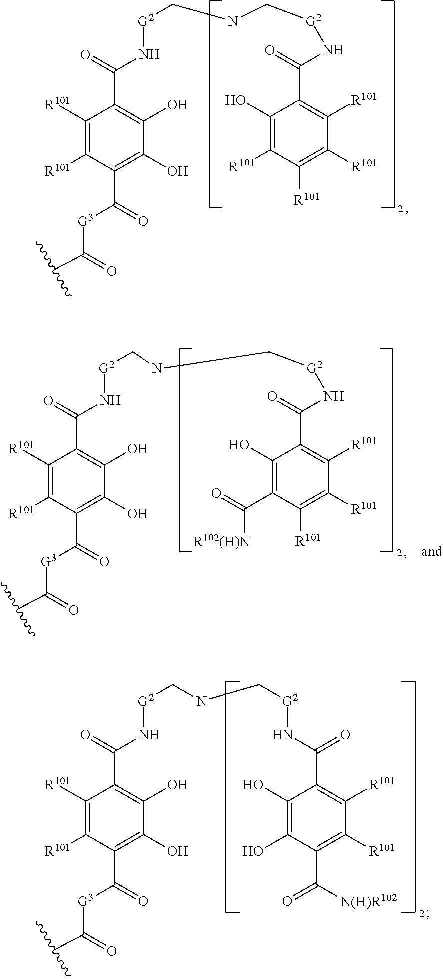
wherein:
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰² is hydrogen, alkyl, alkenyl, alkynyl, cycloalkyl, heterocyclyl, aryl, aralkyl, heteroaryl, heteroaralkyl, or fluoroalkyl
G² is alkylene; and
G³ is alkylamine or dialkylamine, wherein the alkyl moiety is optionally substituted with halogen, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

43. The oligonucleotide of claim 31, wherein Z is selected from the group consisting of: 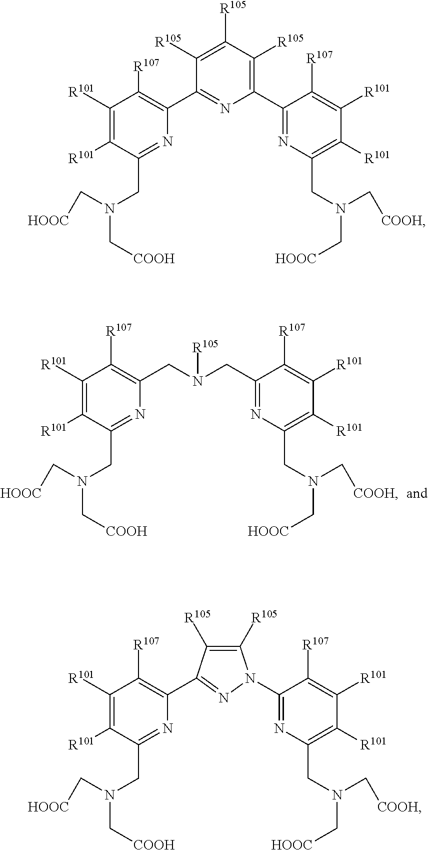
wherein:
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰⁷ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰⁵ is -alkylene-E, -heteroalkylene-E, -alkenylene-E, -alkynylene-E, -arylene-E, or -aralkylene-E; and
E is —(C═O)— or -Ph-N—C(═S)—.

44. The oligonucleotide of claim 31, wherein Z is represented by compound of formula (IX): 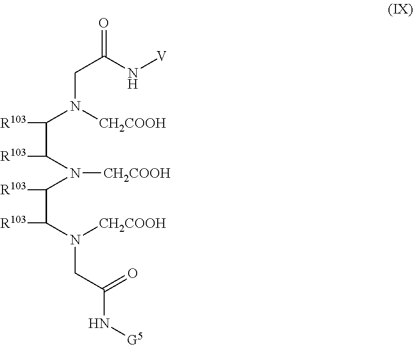
wherein V is an organic chromophore;
G⁵ is -alkylene-E, -heteroalkylene-E, -alkenylene-E, -alkynylene-E, -arylene-E, or -aralkylene-E;
R¹⁰³ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and
E is —(C═O)— or -Ph-N—C(═S)—.

45. The oligonucleotide of claim 31, wherein L² is 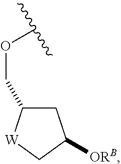
where W is NC(═O)— alkylene-N(H)Z and R^{B} is H.

46. The oligonucleotide of claim 31, wherein M² is present in the oligonucleotide at least once.

47. The oligonucleotide of claim 31, further comprising at least one conjugate.

48. The oligonucleotide of claim 47, wherein the conjugate is selected from the group consisting of cholesterol, cholic acid, hexyl-S-tritylthiol, thiocholesterol, dodecandiol, di-hexadecyl-rac-glycerol, triethylammonium 1,2-di-O-hexadecyl-rac-glycero-3-H-phosphonate, polyamine, polyethylene glycol, adamantane acetic acid, palmityl, octadecylamine, and hexylamino-carbonyl-oxycholesterol.

49. A method of using the oligonucleotide of claim 31 to acquire an image or images in a subject with a diagnostic imaging technique to determine the bio-distribution of said oligonucleotide.

50. A process of preparing the double-stranded oligonucleotide of claim 33, comprising the steps of a) chelating a metal with a metal ligand to form a chelated metal; b) complexing the chelated metal with the complementary guide strand or passenger strand; and c) annealing the metal labeled oligonucleotide of step b) with complementary guide strand or passenger strand.

51. The process of claim 50, wherein the metal is Gd(III), Mn(II), Mn(III), Cr(II), Cr(III), Cu(II), Fe (III), Pr(III), Nd(III), Sm(III), Tb(III), Yb(III) Dy(III), Ho(III), Eu(II), Eu(III), or Er(III).

52. The process of claim 50, wherein the metal ligand is selected from a compound represented by formula (II) or (III): 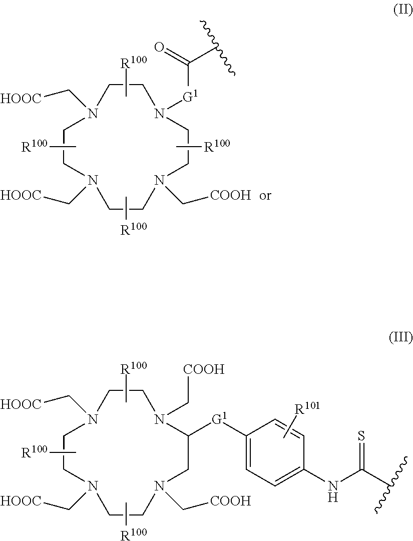
wherein, independently for each occurrence,
G¹ is alkylene, heteroalkylene, alkenylene, alkynylene, arylene, or aralkylene;
R¹⁰⁰ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

53. The process of claim 50, wherein the metal ligand is selected from the group consisting of: 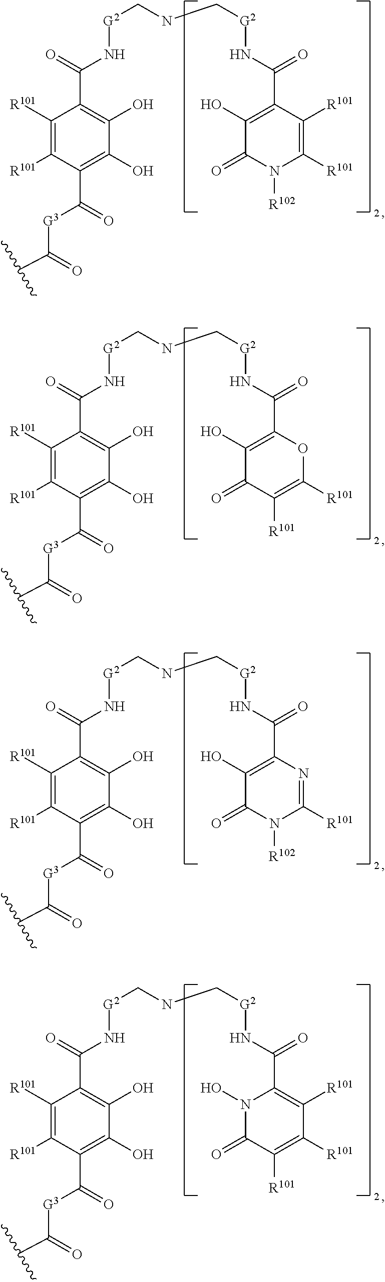 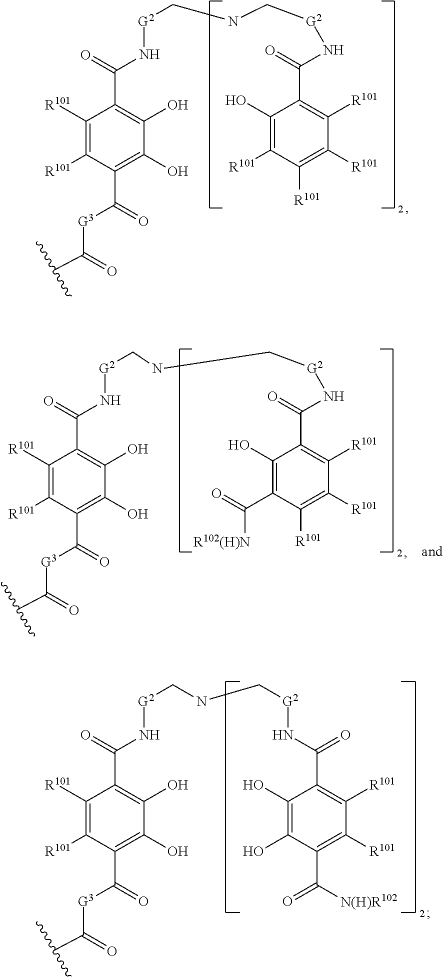
wherein:
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰² is hydrogen, alkyl, alkenyl, alkynyl, cycloalkyl, heterocyclyl, aryl, aralkyl, heteroaryl, heteroaralkyl, or fluoroalkyl;
G² is alkylene; and
G³ is alkylene or heteroalkylene.

54. The process of claim 50, wherein the metal ligand is selected from the group consisting of: 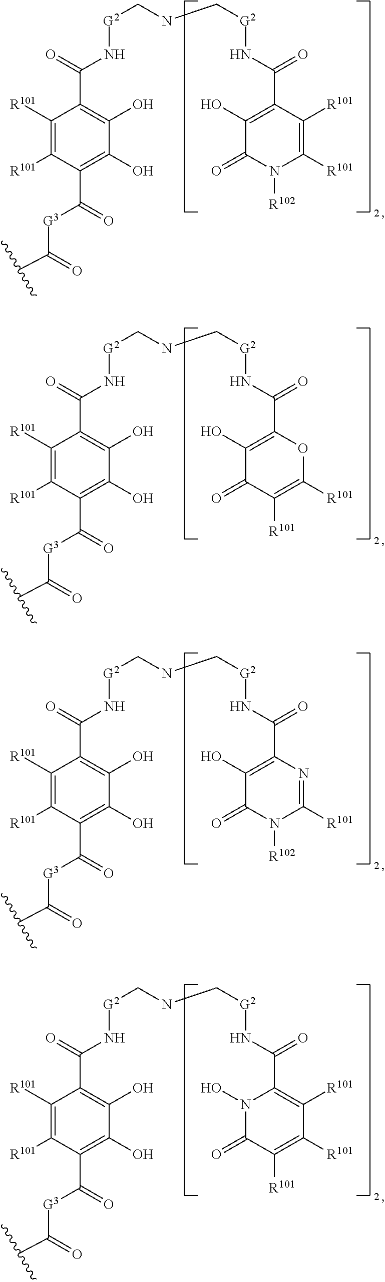 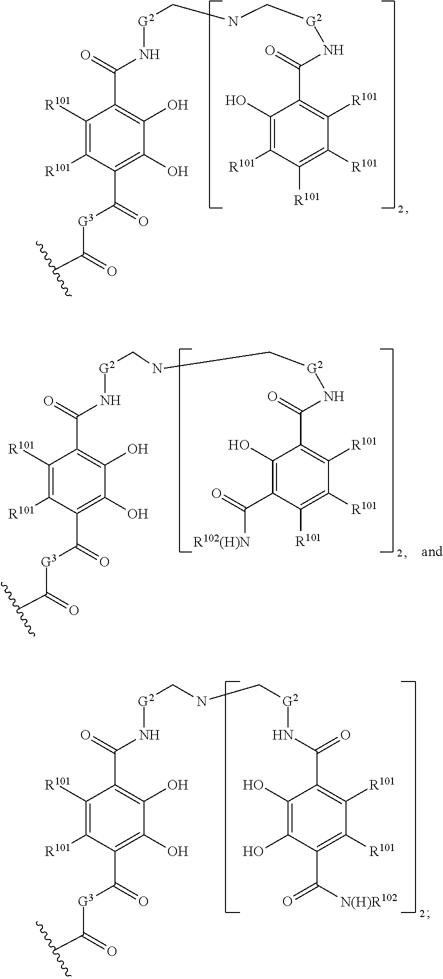
wherein:
R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰² is hydrogen, alkyl, alkenyl, alkynyl, cycloalkyl, heterocyclyl, aryl, aralkyl, heteroaryl, heteroaralkyl, or fluoroalkyl;
G² is alkylene; and
G³ is alkylamine or dialkylamine, wherein the alkyl moiety is optionally substituted with halogen, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

55. The process of claim 50, wherein the metal ligand is selected from the group consisting of: 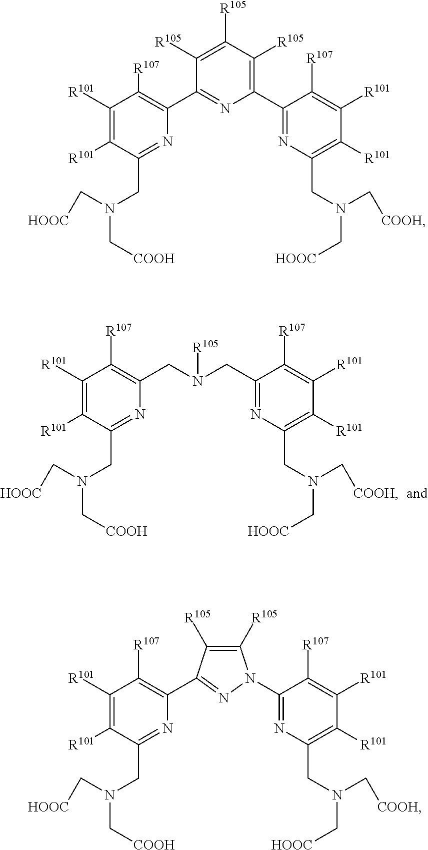
where R¹⁰¹ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰⁷ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano;
R¹⁰⁵ is -alkylene-E, -heteroalkylene-E, -alkenylene-E, -alkynylene-E, -arylene-E, or -aralkylene-E; and
E is —(C═O)— or -Ph-N—C(═S)—.

56. The process of claim 50, wherein the metal ligand is represented by a compound of formula (IX): 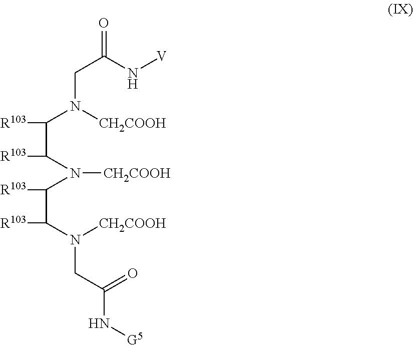
wherein V is an organic chromophore;
G⁵ is -alkylene-E, -heteroalkylene-E, -alkenylene-E, -alkynylene-E, -arylene-E, or -aralkylene-E;
R¹⁰³ is hydrogen, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano; and
E is —(C═O)— or -Ph-N—C(═S)—.

57. The process of claim 50, wherein oligonucleotide comprises 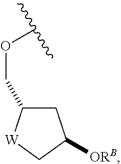
where W is NC(═O)-alkylene-N(H)Z and R^{B} is H.

58. The process of claim 50, wherein M² is present in the oligonucleotide at least once.

59. The process of claim 50, wherein the oligonucleotide further comprises at least one conjugate.

60. The process of claim 59, wherein the conjugate is selected from the group consisting of cholesterol, cholic acid, hexyl-S-tritylthiol, thiocholesterol, dodecandiol, di-hexadecyl-rac-glycerol, triethylammonium 1,2-di-O-hexadecyl-rac-glycero-3-H-phosphonate, polyamine, polyethylene glycol, adamantane acetic acid, palmityl, octadecylamine, and hexylamino-carbonyl-oxycholesterol.


SEQUENCE LISTING (1 sequence)

SEQ ID NO: 1 - DNA, 21 bp, Artificial Sequence
  Description of Combined DNA/RNA Molecule Synthetic oligonucleotide
cuuacgcugaguacuucgatt